(19) United States
(12) Patent Application Publication
(10) Pub. No.: US 2026/0248128 A1
(43) Pub. Date: Aug. 27, 2026

(54) INSECT NEUROPEPTIDE ANALOGUES

(71) Applicant: Solasta Bio Limited, Glasgow (GB)

(72) Inventors: Shireen-Anne Davies, East Dunbartonshire (GB); Julian Alexander Thomas Dow, East Dunbartonshire (GB); Yousef Abul-Haija, Glasgow (GB)

(73) Assignee: Solasta Bio Limited, Glasgow (GB)

(21) Appl. No.: 18/714,954

(22) PCT Filed: Dec. 2, 2022
(86) PCT No.: PCT/GB2022/053082

§ 371 Date: May 30, 2024

(30) Foreign Application Priority Data
Dec. 3, 2021 (GB) 2117537.7
Jun. 9, 2022 (GB) 2208466.9

(51) Int. Cl. A01N 37/46; A01N 25/02; A01P 7/04; C07K 7/08

(52) U.S. Cl. CPC A01N 37/46; A01N 25/02; A01P 7/04; C07K 7/08

(57) ABSTRACT

The present invention relates to analogues of insect neuropeptides having activity against insects, such as hemipteran, dipteran and/or lepidopteran insects, such as aphids and fruit flies, and their use as insect control agents (e.g. insecticides) and plant protection agents.

### FIELD OF THE INVENTION

The present invention relates to analogues of insect neuropeptides having activity against insects, for example hemipteran, dipteran and/or lepidopteran insects, such as aphids and fruit flies, and their use as insect control agents (e.g. insecticides) and plant protection agents.

### BACKGROUND

With a global dependence on broad-spectrum insecticides, the damaging effects of which are well documented, there is increasing need for the development of greener, target-specific insecticides. The development and employment of neuropeptide synthetic analogues offers a promising avenue in the drive for greener and target-specific insecticidal agents. Within insects, neuropeptides are regulatory peptides with functional roles in growth and development, behaviour and reproduction, metabolism and homeostasis, and muscle movement. Due to their high specificity, neuropeptides and their cognate receptors (G-protein coupled receptors, GPCRs) may be developed towards insecticidal agents to selectively reduce the fitness of target pest insects, whilst minimising detrimental environmental impacts.

Insect neuropeptide families include the insect kinins and cardio acceleratory peptides (CAPA, CAP2b) neuropeptides.

The CAPA peptides were first identified from the moth Manduca sexta (CAP2b) and have since been identified in many insect families. Although function varies depending on insect species, life stage, and lifestyle, CAPA peptides play key roles in homeostasis and metabolism (Koyama et al., Nature Communications, 2021).

The CAPA peptides belong to the PRXamide superfamily which can be further subdivided into three major classes: CAPA peptides (Periviscerokinins), pyrokinins (PK) and ecdysis triggering hormone (ETH). CAPA peptides and Pyrokinins in some insects are encoded by the same gene, e.g. in Drosophila melanogaster, CAPA-1, CAPA-2 and CAPA-3 (PK) are all encoded by the CAPA gene (Kean et al., Am J Physiology, 2002).

Hemipteran insect species including M. persicae contain three CAPA peptide sequences, CAPA-1, CAPA-2 and CAPA-3 (see Table 1).

Interrogation of the DiNER database of insect neuropeptides (Yeoh et al., Insect Biochem and Mol Biol, 2018), for CAPA-3 sequences shows that CAPA-3 is conserved within Hemipteran insects, for which sequences are known (Table 2). CAPA-3 is distinct from CAPA-1 and 2 within the Hemiptera and in other insect orders e.g. Diptera and Lepidoptera. Many Dipteran species have only CAPA-1 and CAPA-2 peptides, which are most often found with a C-terminal PRV motif, although in the tsetse fly Glossina morsitans, CAPA-1 and CAPA-2 have a C-terminal PRI motif. The conserved CAPA-3 C-terminal signature in Hemiptera is GPRL. This is also found in the Dipteran pest oriental fruit fly Bactrocera dorsalis, which encodes CAPA-3 in addition to CAPA-1 and CAPA-2. Lepidopteran species encode CAPA-1 and CAPA-2 with the PRV motif but in species which encode CAPA-3, including the pest species Heliothis peltigera and Spodoptera spp., CAPA-3 contains the GPRL motif.

CAPA-3 has been shown to activate a Pyrokinin receptor in Brown Marmorated Stinkbug, H. halys (Ahn et al., 2020).

The present invention provides analogues of CAPA-3 peptides.

### SUMMARY OF THE INVENTION

The inventors have discovered new analogues of CAPA peptides having insecticidal activity against insects that encode the CAPA-3 peptide, such as hemipteran, dipteran and/or lepidopteran insects, and so potentially finding use as pest control agents or insecticides, while having little or no effect against important pollinator species such as bees.

Thus, in a first aspect, the invention provides an insecticidal compound having the formula (I) below:

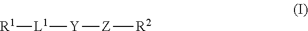

wherein:
R¹ is hydrogen (which may be designated “H-” or “Hy-”), C₁₋₄alkyl (e.g. methyl, ethyl, propyl, butyl), formyl, —N(R^{1a})—C(═N+(R^{1b})(R^{1c}))NR^{1d}R^{1e}, —C(═N⁺—(R^{1b})(R^{1c}))NR^{1d}R^{1e}, acyl, fatty acyl, benzyl, benzoyl, heteroaryl or trifluoroacetyl, —NHC₁₋₁₈alkyl, —NHC₅₋₁₆Aryl, —NH—C₁₋₆alkyl-C₆₋₁₀aryl, biotin, a sugar moiety or (poly)alkyleneglycol;
wherein each of R^{1a}, R^{1b}, R^{1c}, R^{1d} and R^{1e} is independently selected from hydrogen or C₁₋₄ alkyl (e.g. methyl, ethyl, propyl, butyl);
and any alkyl, formyl, acyl, fatty acyl, benzyl, benzoyl, heteroaryl or trifluoroacetyl, —NHC₁₋₁₈alkyl, —NHC₆₋₁₆Aryl, —NH—C₁₋₆alkyl-C₆₋₁₀aryl or (poly)alkyleneglycol may optionally be substituted with one or more groups selected from halogen, C₁₋₆alkyl, C₁₋₆haloalkyl or a sugar moiety;
L¹ is absent or is:
   *—(C═O)C₁₋₁₆-alkylene-NH— where * denotes the point of attachment to Z;
   (C₁₋₂₀)alkylene,
   (C₂₋₂₀)alkenylene,
   (poly)alkyleneglycol;
   wherein L¹ is optionally substituted with one or more groups selected from oxo (═O), halogen, ═N and ═S;
Z^{a} is peptide comprising from 1 to 12 amino acids, wherein Z^{a} optionally comprises a (poly)alkyleneglycol linker in the peptide sequence;
Z is a peptide of the formula:

SEQ ID NO: 123
W^{#}-F^{#}-G^{#}-P^{#}-R^{#}-L^{#}

wherein “^{#}” indicates that the residues “W^{#}”, “F^{#}”, “G^{#}”, “P^{#}”, “R^{#}” and “L^{#}” are independently an unmodified amino acid, a modified amino acid or non-natural amino acid analogue; and
at least one of the residues in peptides Z^{a} and Z is a modified amino acid or non-natural amino acid analogue; and
R² is NH₂, NR^{2a}H, NR^{2a}R^{2b}, or OR^{2a}; wherein each of R^{2a} and R^{2b} is independently C₁₋₆-alkyl (e.g. methyl, ethyl, propyl, butyl, pentyl or hexyl), C₃₋₆-alkenyl, C₆₋₁₆-aryl, C₆₋₁₆-aryl-C₁₋₆-alkyl, C₁₋₆-alkylene-C₆₋₁₆-aryl, or C₁₋₆-haloalkyl, each of which may optionally be substituted with one or more groups selected from halogen, C₁₋₆-alkyl, or C₁₋₆-haloalkyl.

In another aspect, there is provided a composition comprising a compound as defined herein, or a salt thereof, in admixture with one or more solvents, carriers, diluents, adjuvants, preservatives, dispersants, emulsifying agents, or synergists. Suitably, the composition is an insect control composition or plant protection composition.

In another aspect, there is provided the use of a compound as defined herein, or a salt thereof, or a composition as defined herein, as an insect control agent. The use may be as an insecticide against insects that encode the CAPA-3 peptide. The use may be as an insecticide against hemipteran, dipteran and/or lepidopteran insects.

In another aspect, there is provided a method of insect mortality, the method comprising contacting an insect population with a compound as defined herein, or a salt thereof, or a composition as defined herein. Suitably, the insect is an insect that encodes the CAPA-3 peptide, such as hemipteran, dipteran and/or lepidopteran insect mortality.

In another aspect, there is provided a method of increasing hemipteran, dipteran and/or lepidopteran insect mortality, the method comprising contacting a hemipteran insect or hemipteran insect population with a compound as defined herein, or a salt thereof, or a composition as defined herein.

In another aspect, there is provided the use of a compound as defined herein, or a salt thereof, or a composition as defined herein, as a plant protection agent, for protecting a plant against insects that encode the CAPA-3 peptide.

In another aspect, there is provided the use of a compound as defined herein, or a salt thereof, or a composition as defined herein, as a plant protection agent, for protecting a plant against hemipteran, dipteran and/or lepidopteran insects.

In another aspect, there is provided a method of inhibiting infestation of a plant by insects that encode the CAPA-3 peptide, the method comprising contacting the plant with a compound as defined herein, or a salt thereof, or a composition as defined herein. Suitably, the compound or composition is applied to the plant while the plant is free or substantially free of insects that encode the CAPA-3 peptide.

In another aspect, there is provided a method of inhibiting infestation of a plant by hemipteran, dipteran and/or lepidopteran insects comprising contacting the plant with a compound as defined herein, or a salt thereof, or a composition as defined herein. Suitably, the compound or composition is applied to the plant while the plant is free or substantially free of hemipteran, dipteran and/or lepidopteran insects.

In another aspect, there is provided a method of reducing infestation of a plant by insects that encode the CAPA-3 peptide, or of reducing load of insects that encode the CAPA-3 peptide on a plant, the method comprising contacting the plant with a compound as defined herein, or a salt thereof, or a composition as defined herein.

In another aspect, there is provided a method of reducing hemipteran, dipteran and/or lepidopteran insect infestation of a plant, or of reducing hemipteran, dipteran and/or lepidopteran insect load on a plant, the method comprising contacting the plant with a compound as defined herein, or a salt thereof, or a composition as defined herein.

### DETAILED DESCRIPTION OF THE INVENTION

### Compounds of The Invention

Particular compounds of the invention include, for example, compounds of the formula (I), wherein, unless otherwise stated, each of R¹, L¹, Z^{a}, Z, R² and any associated substituent group has any of the meanings defined hereinbefore or in any of paragraphs (1) to (26) hereinafter:—
(1) R¹ is selected from:
   hydrogen
   —C(═N*Me₂)NMe₂,
   acyl,
   fatty acyl,
   benzyl,
   benzoyl,
   heteroaryl
   trifluoroacetyl,
   —NHC₁₋₁₈alkyl,
   —NHC₆₋₁₆Aryl,
   —NH—C₁₋₆alkyl-C₆₋₁₀aryl;
   a sugar moiety;
   biotin;
   (poly)alkyleneglycol (e.g. polyethylene glycol such as PEG-1 to PEG-16); wherein each of R^{1a}, R^{1b}, R^{1c}, R^{1d} and R^{1e} is independently selected from hydrogen or C₁₋₄ alkyl (e.g. methyl, ethyl, propyl, butyl);
   and any alkyl, formyl, acyl, fatty acyl, benzyl, benzoyl, heteroaryl or trifluoroacetyl, —NHC₁₋₁₈alkyl, —NHC₆₋₁₆-Aryl, —NH—C₁₋₆alkyl-C₆₋₁₀aryl or (poly)alkyleneglycol may optionally be substituted with one or more groups selected from halogen, C₁₋₆alkyl, C₁₋₆haloalkyl or a sugar moiety;
(2) R¹ is selected from:
   hydrogen;
   acyl optionally substituted with a sugar moiety;
   fatty acyl;
   heteroaryl;
   —NHC₆₋₁₆Aryl;
   a sugar moeity;
   biotin; or
   (poly)ethyleneglycol of the formula *—(OCH₂CH₂)ₙ—Rₚ, where * denotes the point of attachment to L¹ or Z, n is an integer from 1 to 16, and Rₚ is selected from —NH₂, —OH or —OMe.
(3) R¹ is selected from:
   hydrogen;
   acyl optionally substituted with a sugar moiety, wherein the acyl group is selected from formyl, acetyl (Ac), propanoyl, butanoyl;
   fatty acyl selected from palmitoyl, butyryl, cerotoyl, decanoyl, docosenoyl, dodecanoyl, eleostearoyl, heptanoyl, hexanoyl, icosanoyl, icosenoyl, lignoceroyl, linoleoyl, lipoyl, myristoleoyl, nonanoyl, octadecanoyl, ocatanoyl, palmitoleoyl, stearoyl, undecanoyl, and valeryl;
   heteroaryl selected from indolyl, e.g. 3-indolyl:

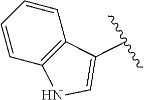 [SMILES: *c1c[nH]c2ccccc12]

—NHC₆₋₁₆Aryl;
a monosaccharide or disaccharide moiety;
biotin;
(poly)ethyleneglycol of the formula *—(OCH₂CH₂)ₙ—Rₚ, where * denotes the point of attachment to L¹ or Z, n is an integer from 4 to 12, and Rₚ is selected from —NH₂, —OH or —OMe.
(4) R¹ is selected from:
   hydrogen;
   acetyl substituted with a monosaccharide or disaccharide moiety;
   palmitoyl;
   indolyl, e.g. 3-indolyl:

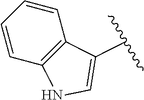 [SMILES: *c1c[nH]c2ccccc12]

—N(H)-fluorenyl substituted with one or more bromine atoms, e.g.

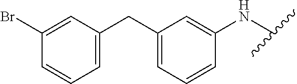 [SMILES: *Nc1cccc(Cc2cccc(Br)c2)c1]

(poly)ethyleneglycol of the formula *—(OCH₂CH₂)ₙ—Rₚ, where * denotes the point of attachment to Z, n is an integer from 6 to 10, and Rₚ is selected from —NH₂, -or OH.
(5) R¹ is selected from:
   hydrogen;
   indolyl, e.g. 3-indolyl:

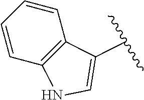 [SMILES: *c1c[nH]c2ccccc12]

(poly)ethyleneglycol of the formula *—(OCH₂CH₂)₈—NH₂ (PEG8), where * denotes the point of attachment to Z.
(6) R¹ is hydrogen.
(7) L¹ is absent or is:
   *—(C═O)C₁₋₁₀-alkylene-NH—, where * denotes the point of attachment to Z; C₁₋₁₀alkylene,
   wherein L¹ is optionally substituted with one or more oxo (═O) groups.
(8) L¹ is absent or is:
   *—(C═O)C₄₋₁₂-alkylene-NH—,
   —(C═O)—C₁₋₄alkylene-(C═O)—,
   *—(C═O)—C₁₋₄alkylene;
   where * denotes the point of attachment to Z.
(9) L¹ is absent or is:

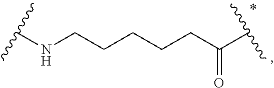 [SMILES: *NCCCCCC(*)=O]

where * denotes the point of attachment to Z;

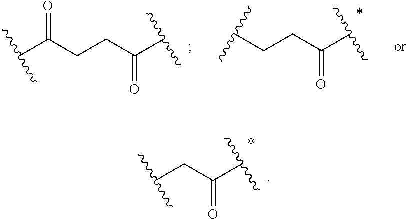 [SMILES: *C(=O)CCC(*)=O ; *CCC(*)=O ; *CC(*)=O]

(10) L¹ is absent.
(11) Either:
   (A) R¹ is selected from:
      (i) hydrogen;
      (ii) acyl optionally substituted with a monosaccharide or disaccharide moiety;
      (iii) fatty acyl;
      (iv) heteroaryl; or
      (v) (poly)ethyleneglycol of the formula *—(OCH₂CH₂)ₙ—NH₂ or *—(OCH₂—CH₂)ₙ—OH where * denotes the point of attachment to L¹ or Z, and n is an integer from 1 to 16; and
      L¹ is absent or is *—(C═O)—C₁₋₄alkylene;
   (B) R¹ is hydrogen, and L¹ is selected from:
      (i) *—(C═O)C₁₋₁₆-alkylene-NH₂, where * denotes the point of attachment to Z;
      (ii) C₄₋₁₄alkylene
      wherein L¹ is optionally substituted with one or more oxo (═O) groups; or
   (C) R¹ is —NHC₆₋₁₆Aryl and L¹ is *—(C═O)C₁₋₁₀-alkylene-NH—.
(12) Either:
   (A) R¹ is selected from:
      (i) hydrogen;
      (ii) acetyl substituted with a monosaccharide or disaccharide moiety;
      (iii) palmitoyl;
      (iv) indolyl; and
      (v) (poly)ethyleneglycol of the formula *—(OCH₂CH₂)₈—NH₂, where * denotes the point of attachment to Z; and
         L¹ is absent or is:

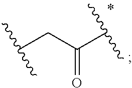 [SMILES: *CC(*)=O]

(B) R¹-L¹ is selected from:
   (i) *—(C═O)C₅alkylene-NH₂, where * denotes the point of attachment to Z, i.e.

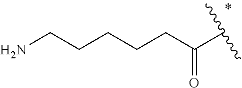 [SMILES: *C(=O)CCCCCN]

(6-aminohexanoic acid)
(ii) *—(C═O)C₁₁alkylene-NH₂, where * denotes the point of attachment to Z, i.e.

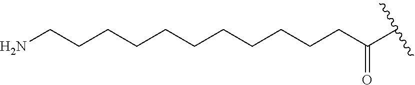 [SMILES: *C(=O)CCCCCCCCCCCN]

(12-aminododecanoic acid)
(iii) a group 2Abf-Suc, i.e.:

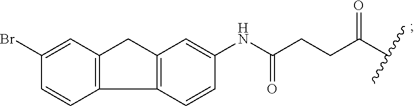 [SMILES: *C(=O)CCC(=O)Nc1ccc2c(c1)Cc1cc(Br)ccc1-2]

(iv) a 3-indoleacetic acid group, e.g.:

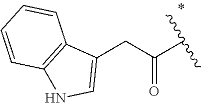 [SMILES: *C(=O)Cc1c[nH]c2ccccc12]

(3-indoleacetic acid, given the notation [ind] in the sequences defined herein)
(13) R¹ is selected from:
   (i) hydrogen;
   (ii) indolyl; and
   (iii) (poly)ethyleneglycol of the formula *—(OCH₂CH₂)₈—NH—, where * denotes the point of attachment to Z; and
   L¹ is absent;
(14) Z^{a} is a peptide containing from 4 to 10 amino acids, optionally comprising a PEG linker therein.
(15) Z^{a} is a peptide containing 7 to 9 amino acids.
(16) Z^{a} is a peptide containing 8 amino acids.
(17) Z^{a} is a peptide of the formula:

SEQ ID NO: 124
S^{#}-D^{#}-S^{#}-K^{#}-N^{#}-T^{#}-A^{#}-L^{#}-

wherein “^{#}” indicates that the residue is either a naturally occurring amino acid, modified amino acid or a non-natural amino acid analogue.
(17a) Z^{a} is a peptide of the formula:
   wherein “^{#}” indicates that the residue is either a naturally occurring amino acid, modified amino acid or a non-natural amino acid analogue.
(18) The Z^{a} moiety has the formula:

| | |
|---|---|
| SDSKNTAL | SEQ ID 1 |
| SDSKNT[n-me-A]L | SEQ ID 2 |
| SDSKNT[Peptoid-Phe]L | SEQ ID 3 |
| SDSKNT[Peptoid-Ala]L | SEQ ID 4 |
| SDSKNTA[Peptoid-Leu] | SEQ ID 5 |
| SDSKNTA[n-me-L] | SEQ ID 6 |
| [glucosamine-S]DSKNTAL | SEQ ID 7 |
| SD[glucosamine-S]KNTAL | SEQ ID 8 |
| SDSK[glucosamine-N]TAL | SEQ ID 9 |
| [glucosamine-Suc]-SDSKNTAL | SEQ ID 10 |
| [glucosamine-Suc]-SDSKNT[n-me-A]L | SEQ ID 11 |
| [Biotin]-SDSKNTAL | SEQ ID 12 |
| SDS[Biotin-K]NTAL | SEQ ID 13 |
| S[Biotin-D]SKNTAL | SEQ ID 153 |
| [Biotin]-SDSKNT[n-me-A]L | SEQ ID 15 |
| SDS[Biotin-K]NT[n-me-A]L | SEQ ID 16 |
| S[Biotin-D]SKNT[n-me-A]L | SEQ ID 154 |
| SDSKNT[Aib]L | SEQ ID 18 |
| SDSKNT[a]L; | SEQ ID 19 |
| SDSKNTA[I]; | SEQ ID 20 |
| SDSKNT[a][I] | SEQ ID 21 |
| SDSKNT[a]L | SEQ ID 22 |
| SDSKNTA[I] | SEQ ID 23 |
| SDSKNT[a][I]; | SEQ ID 24 |
| SDSKNT[B3A]L; | SEQ ID 25 |
| SDSKNTA[B3L]; | SEQ ID 26 |
| SDSKNT[Aib]L; or | SEQ ID 27 |
| L. | SEQ ID 28 |

(18a) The Z^{a} moiety has the formula:

| | |
|---|---|
| SDSKNTAL | SEQ ID 1 |
| SDSKNT[n-me-A]L | SEQ ID 2 |
| SDSKNT[Peptoid-Phe]L | SEQ ID 3 |
| SDSKNT[Peptoid-Ala]L | SEQ ID 4 |
| SDSKNTA[Peptoid-Leu] | SEQ ID 5 |
| SDSKNTA[n-me-L] | SEQ ID 6 |
| [glucosamine-S]DSKNTAL | SEQ ID 7 |
| SD[glucosamine-S]KNTAL | SEQ ID 8 |
| SDSK[glucosamine-N]TAL | SEQ ID 9 |
| SDS[Biotin-K]NTAL | SEQ ID 13 |
| S[Biotin-PEG-D]SKNTAL | SEQ ID 14 |
| SDS[Biotin-K]NT[n-me-A]L | SEQ ID 16 |
| S[Biotin-PEG-D]SKNT[n-me-A]L | SEQ ID 17 |
| SDSKNT[Aib]L | SEQ ID 18 |
| SDSKNT[a]L; | SEQ ID 19 |
| SDSKNTA[I]; | SEQ ID 20 |
| SDSKNT[a][I] | SEQ ID 21 |
| SDSKNT[a]L | SEQ ID 22 |
| SDSKNTA[I] | SEQ ID 23 |
| SDSKNT[a][I]; | SEQ ID 24 |
| SDSKNT[B3A]L; | SEQ ID 25 |
| SDSKNTA[B3L]; | SEQ ID 26 |
| SDSKNT[Aib]L; or | SEQ ID 27 |
| L. | SEQ ID 28 |

(19) The Z moiety has the formula:

| | |
|---|---|
| WFGPRL; | SEQ ID 29 |
| W[Peptoid Phe]GPRL; | SEQ ID 30 |
| W[n-me-F]GPRL; | SEQ ID 31 |
| WFGPR[n-me-L]; | SEQ ID 32 |
| W[f]GPRL; | SEQ ID 33 |
| [w]FGPRL; | SEQ ID 34 |
| [w][f]GPRL; | SEQ ID 35 |
| W[B3F]GPRL; or | SEQ ID 36 |
| WFG[Hyp]RL. | SEQ ID 37 |

(19a) The Z moiety has the formula:

| | |
|---|---|
| WFGPRL; | SEQ ID 29 |
| W[Peptoid Phe]GPRL; | SEQ ID 30 |
| W[n-me-F]GPRL; | SEQ ID 31 |
| WFGPR[n-me-L]; | SEQ ID 32 |
| W[f]GPRL; | SEQ ID 33 |
| [w]FGPRL; | SEQ ID 34 |
| [w][f]GPRL; | SEQ ID 35 |
| W[B3F]GPRL; | SEQ ID 36 |
| WFG[Hyp]RL; or | SEQ ID 37 |
| W-[Phg]-[Sar]-PR-[n-me-L]. | SEQ ID 38 |

(20) The Z moiety has the formula WFGPRL; (SEQ ID 29)
(20a) At least one of the residues in peptides Z^{a} and Z is:
   i. an N-methylated amino acid;
   ii. a D-configuration amino acid;
   iii. a beta-configuration amino acid
   iv. a peptoid amino acid analogue;
   v. a sugar modified amino acid;
   vi. a biotin modified amino acid;
   vii. a non-proteinogenic amino acid, such as hydroxyproline (Hyp: L-hydroxyproline or (2S,4R)-4-Hydroxyproline), Octahydroindole-2-carboxylic acid (Oic), sarcosine (Sar), norleucine (Nie), α-aminoisobutyric acid (Aib), thiazolidine-4-carboxylic acid (Thz) or phenylglycine (Phg).
(21) At least one of the residues in peptides Z^{a} and Z is:
   i. an N-methylated amino acid;
   ii. a D-configuration amino acid;
   iii. a beta-configuration amino acid
   iv. a peptoid amino acid analogue;
   v. a sugar modified amino acid;
   vi. a biotin modified amino acid.
(21a) At least one of the residues in peptides Z^{a} and Z is:
   i. an N-methylated amino acid;
   ii. a D-configuration amino acid;
   iii. a beta-configuration amino acid
   iv. a peptoid amino acid analogue;
   v. a sugar modified amino acid;
   vi. a biotin modified amino acid;
   vii. hydroxyproline (Hyp);
   viii. sarcosine (Sar);
   ix. α-aminoisobutyric acid (Aib); or
   x. phenylglycine (Phg).
(22) At least one of the residues in peptides Z^{a} and Z is:
   i. an N-methylated amino acid;
   ii. a peptoid amino acid analogue;
   iii. a sugar modified amino acid;
   iv. a biotin modified amino acid.
(23) At least one of the residues in peptides Z^{a} and Z is:
   i. an N-methylated amino acid;
   ii. a peptoid amino acid analogue.
(24) At least one of the residues in peptides Z^{a} and Z is an N-methylated amino acid.
(25) R² is NH₂, NR^{2a}H or NR^{2a}R^{2b}, wherein each of R^{2a} and R²ⁿ if present is independently C₁₋₆-alkyl (e.g. methyl, ethyl, propyl, butyl, pentyl or hexyl).
(26) R₂ is NH₂.

Suitably, R¹ is as defined in any one of paragraphs (1) to (6) above. Most suitably, R¹ is as defined in any one of paragraphs (4) to (6) above.

Suitably, L¹ is as defined in any one of paragraphs (7) to (10) above. Most suitably, L¹ is as defined in paragraph (9) or (10) above.

Suitably, R¹ and L¹ are as defined in any one of paragraphs (11) to (13) above. Most suitably, R¹ and L¹ are as defined in paragraph (13) above.

Suitably, Z^{a} is as defined in any one of paragraphs (14) to (18a) above. Most suitably, Z^{a} is as defined in any one of paragraphs (17), (17a), (18) or (18a) above. Most suitably, Z^{a} is as defined in any one of paragraphs (17) or (18) above. Suitably, Z^{a} is as defined in any one of paragraphs (17a or (18a) above.

In an embodiment, Z^{a} is as defined in paragraph (17), i.e. Z^{a} is a peptide of the formula:

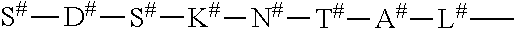

and the peptide Z^{a} comprises no more than three modified amino acid or non-natural amino acid analogues. Suitably, the peptide Z^{a} comprises no more than two modified amino acid or non-natural amino acid analogues. Suitably, the peptide Z^{a} comprises only one modified amino acid or non-natural amino acid analogue.

Suitably, Z is as defined in any one of paragraph (19) or (20) above.

Suitably, the at least one modified amino acid or non-natural amino acid analogue in peptide Z and/or Z^{a} is as defined in any one of paragraphs (20a), (21), (21a), (22), (23) or (24) above. Suitably, the at least one modified amino acid or non-natural amino acid analogue in peptide Z and/or Z^{a} is as defined in any one of paragraphs (21) to (24) above. More suitably, the at least one modified amino acid or non-natural amino acid analogue in peptide Z and/or Z^{a} is as defined in any one of paragraphs (22) to (24) above. Most suitably, the at least one modified amino acid or non-natural amino acid analogue in peptide Z and/or Z^{a} is as defined in paragraph (24), i.e. it is an N-methylated amino acid.

Suitably, R₂ is as defined in paragraph (25) or (26) above. Most suitably, R₂ is as defined in paragraph (26) above.

In an embodiment, Z is a peptide with a formula selected from:

SEQ ID NO: 125
W^{#}-F-G-P-R-L
SEQ ID NO: 126
W-F^{#}-G-P-R-L
SEQ ID NO: 127
W-F-G-P^{#}-R-L
SEQ ID NO: 128
W-F-G-P-R^{#}-L
SEQ ID NO: 129
W-F-G-P-R-L^{#}
SEQ ID NO: 130
W^{#}-F^{#}-G-P-R-L
SEQ ID NO: 131
W^{#}-F-G^{#}-P-R-L
SEQ ID NO: 132
W^{#}-F-G-P^{#}-R-L
SEQ ID NO: 133
W^{#}-F-G-P-R^{#}-L
SEQ ID NO: 134
W^{#}-F-G-P-R-L^{#}
SEQ ID NO: 135
W-F^{#}-G^{#}-P-R-L
SEQ ID NO: 136
W-F^{#}-G-P^{#}-R-L
SEQ ID NO: 137
W-F^{#}-G-P-R^{#}-L
SEQ ID NO: 138
W-F^{#}-G-P-R-L^{#}
SEQ ID NO: 139
W-F-G^{#}-P^{#}-R-L
SEQ ID NO: 140
W-F-G^{#}-P-R^{#}-L
SEQ ID NO: 141
W-F-G^{#}-P-R-L^{#}
SEQ ID NO: 142
W-F-G-P^{#}-R^{#}-L
SEQ ID NO: 143
W-F-G-P^{#}-R-L^{#}
SEQ ID NO: 144
W-F_{#}-G#-P-R-L_{#}

wherein “^{#}” in the formula above indicates that the labelled residue, i.e. “V^{#}”, “F^{#}”, “G^{#}”, “P^{#}”, “R^{#}” or “L^{#}” are independently an unmodified amino acid, a modified amino acid or non-natural amino acid analogue. Suitably, the residues marked with a “^{#}” in the formulas above are a modified amino acid or non-natural amino acid analogue.

As described herein, Z is a peptide of the formula:

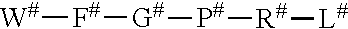

wherein “^{#}” indicates that the residues “W^{#}”, “F^{#}”, “G^{#}”, “P^{#}”, “R^{#}” and “L^{#}” are independently an unmodified amino acid, a modified amino acid or non-natural amino acid analogue.

In an embodiment, the peptide Z comprises five unmodified amino acid residues, and a single modified amino acid or non-natural amino acid analogue as defined herein.

In an embodiment, the peptide Z comprises four unmodified amino acid residues, and two modified amino acid or non-natural amino acid analogues.

In an embodiment the peptide Z comprises three unmodified amino acid residues, and three modified amino acid or non-natural amino acid analogues.

In an embodiment, the peptide Z comprises two unmodified amino acid residues, and four modified amino acid or non-natural amino acid analogues.

In an embodiment, Z is a peptide of the formula:

SEQ ID NO: 149
W^{#}-F^{#}-G^{#}-P^{#}-R^{#}-L^{#}

wherein “^{#}” indicates that the residues “W^{#}”, “F^{#}”, “G^{#}”, “P^{#}”, “R^{#}” and “L^{#}” are independently an unmodified amino acid, a modified amino acid or non-natural amino acid analogue; and
the residue marked W^{#} is selected from W or [w];
the residue marked F^{#} is selected from F, [Peptoid Phe], [n-me-F], [f], [B3F] or [Phg];
the residue marked G^{#} is selected from G or [Sar];
the residue marked P^{#} is selected from P or [Hyp];
the residue marked R^{#} is R;
the residue marked L^{#} is selected from L or [n-me-L]
at least one of the residues in peptides Z^{a} and Z is a modified amino acid or non-natural amino acid analogue.

In an embodiment, Z is a peptide of the formula:

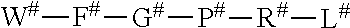

wherein “^{#}” indicates that the residues “W^{#}”, “F^{#}”, “G^{#}”, “P^{#}”, “R^{#}” and “L^{#}” are independently an unmodified amino acid, a modified amino acid or non-natural amino acid analogue; and
the residue marked W^{#} is selected from W or [w];
the residue marked F^{#} is selected from F, [Peptoid Phe], [n-me-F], [f], [B3F] or [Phg];
the residue marked G^{#} is selected from G or [Sar];
the residue marked P^{#} is selected from P or [Hyp];
the residue marked R^{#} is R;
the residue marked L^{#} is selected from L or [n-me-L]; and
at least one of the residues in peptides Z^{a} and Z is a modified amino acid or non-natural amino acid analogue;
wherein no more than 3 amino acid residues in the peptide Z are a modified amino acid or non-natural amino acid analogue. Suitably, no more than 2 amino acid residues in the peptide Z are a modified amino acid or non-natural amino acid analogue. Suitably, only a single amino acid residue in the peptide Z is a modified amino acid or non-natural amino acid analogue.

In a preferred embodiment, Z is a peptide of the formula:

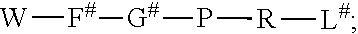

the residues marked F^{#}, G^{#} and L^{#} are modified or non-natural amino acid analogues as described herein.

Preferably, at least two of the residues in the peptide Z are the unmodified amino acid. More preferably, at least three of the residues in the peptide Z are the unmodified amino acid.

In certain embodiments, the peptide Z^{a}—Z comprises 1, 2, 3 or 4 modified amino acids or non-natural amino acid analogues. In certain embodiments, the peptide Z^{a}—Z comprises 1, 2 or 3 modified amino acids or non-natural amino acid analogues. In certain embodiments, the peptide Z^{a}—Z comprises 1 or 2 modified amino acids or non-natural amino acid analogues. In certain embodiments, the peptide Z^{a}—Z comprises a single modified amino acid or non-natural amino acid analogue.

In certain embodiments, the peptide Z^{a}—Z comprises no more than 5 modified amino acids. In certain embodiments, the peptide Z^{a}—Z comprises no more than 4 modified amino acids. In certain embodiments, the peptide Z^{a}—Z comprises no more than 3 modified amino acids.

In an embodiment, Z^{a} as defined in paragraph (17), i.e. is a peptide of the formula:

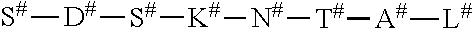

Thus, in a particular group of compounds of the invention the compounds are of the formula (Ia) below:

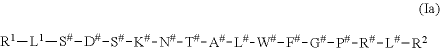

In an embodiment of the compounds of Formula (Ia):

R¹ is as defined in any one of paragraphs (1) to (6) above;
L¹ is as defined in any one of paragraphs (7) to (10) above;
Z^{a} is as defined in any one of paragraphs (14) to (18a) above;
the at least one modified amino acid or non-natural amino acid analogue in peptide Z and/or Z^{a} is as defined in any one of paragraphs (21) to (24) above; and
R₂ is as defined in paragraph (25) or (26) above.

In an embodiment of the compounds of Formula (Ia):
R¹ and L¹ are as defined in paragraph (13) above;
Z^{a} is as defined in paragraph (17) or (18) above;
the at least one modified amino acid or non-natural amino acid analogue in peptide Z and/or Z^{a} is as defined in any one of paragraphs (22) to (24) above; and
R₂ is as defined in paragraph (26) above.

In an embodiment of the compounds of Formula (Ia):
R¹ and L¹ are as defined in paragraph (13) above;
Z^{a} is as defined in paragraph (17a) or (18a) above;
the at least one modified amino acid or non-natural amino acid analogue in peptide Z and/or Z^{a} is as defined in any one of paragraphs (22) to (24) above; and
R₂ is as defined in paragraph (26) above.

In an embodiment of the compounds of Formula (Ia):
R¹ is as defined in any one of paragraphs (1) to (6) above;
L¹ is as defined in any one of paragraphs (7) to (10) above;
Z^{a} is as defined in any one of paragraphs (14) to (18a) above;
the at least one modified amino acid or non-natural amino acid analogue in peptide Z and/or Z^{a} is as defined in any one of paragraphs (21) to (24) above;
R₂ is as defined in paragraph (25) or (26) above; and
   the residue marked W^{#} is selected from W or [w];
   the residue marked F^{#} is selected from F, [Peptoid Phe], [n-me-F], [f], [B3F] or [Phg];
   the residue marked G^{#} is selected from G or [Sar];
   the residue marked P^{#} is selected from P or [Hyp];
   the residue marked R^{#} is R; and the residue marked L^{#} is selected from L or [n-me-L].

In an embodiment of the compounds of Formula (Ia):
R¹ and L¹ are as defined in paragraph (13) above;
Z^{a} is as defined in paragraph (17) or (18) above;
the at least one modified amino acid or non-natural amino acid analogue in peptide Z and/or Z^{a} is as defined in any one of paragraphs (22) to (24) above;
R² is as defined in paragraph (26) above;
   the residue marked W^{#} is selected from W or [w];
   the residue marked F^{#} is selected from F, [Peptoid Phe], [n-me-F], [f], [B3F] or [Phg];
   the residue marked G^{#} is selected from G or [Sar];
   the residue marked P^{#} is selected from P or [Hyp];
   the residue marked R^{#} is R; and
   the residue marked L^{#} is selected from L or [n-me-L].

In an embodiment of the compounds of Formula (Ia):
R¹ and L¹ are as defined in paragraph (13) above;
Z^{a} is as defined in paragraph (17a) or (18a) above;
the at least one modified amino acid or non-natural amino acid analogue in peptide Z and/or Z^{a} is as defined in any one of paragraphs (22) to (24) above;
R² is as defined in paragraph (26) above;
   the residue marked W^{#} is selected from W or [w];
   the residue marked F^{#} is selected from F, [Peptoid Phe], [n-me-F], [f], [B3F] or [Phg];
   the residue marked G^{#} is selected from G or [Sar];
   the residue marked P^{#} is selected from P or [Hyp];
   the residue marked R^{#} is R; and
   the residue marked L^{#} is selected from L or [n-me-L].

In an embodiment, Z^{a} is a peptide of the formula:

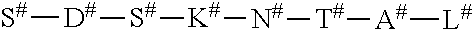

and Z is a peptide of the formula:

(SEQ ID 29)
W-F-G-P-R-L

Thus, in a particular group of compounds of the invention the compounds are of the formula (Ib) below:

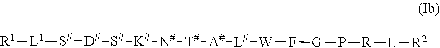

In an embodiment of the compounds of Formula (Ib):
R¹ is as defined in any one of paragraphs (1) to (6) above;
L¹ is as defined in any one of paragraphs (7) to (10) above;
Z^{a} is as defined in any one of paragraphs (14) to (18a) above;
the at least one modified amino acid or non-natural amino acid analogue in peptide Z and/or Z^{a} is as defined in any one of paragraphs (21) to (24) above; and
R₂ is as defined in paragraph (25) or (26) above.

In an embodiment of the compounds of Formula (Ia):
R¹ and L¹ are as defined in paragraph (13) above;
Z^{a} is as defined in paragraph (17) or (18) above;
the at least one modified amino acid or non-natural amino acid analogue in peptide Z and/or Z^{a} is as defined in any one of paragraphs (22) to (24) above; and
R₂ is as defined in paragraph (26) above.

In an embodiment of the compounds of Formula (Ia):
R¹ and L¹ are as defined in paragraph (13) above;
Z^{a} is as defined in paragraph (17a) or (18a) above;
the at least one modified amino acid or non-natural amino acid analogue in peptide Z and/or Z^{a} is as defined in any one of paragraphs (22) to (24) above; and
R₂ is as defined in paragraph (26) above.

### Modified Amino Acid or Non-Natural Amino Acid Analogue

At least one of the residues in peptides Z^{a} and Z is a modified or non-natural amino acid analogue. The modified or non-natural amino acid analogue may be any analogue known to a skilled person. Examples of modified or non-natural amino acid analogues include:
i. N-methylated amino acid;
ii. a peptoid analogue;
iii. a sugar modified analogue;
iv. a biotin modified analogue
v. beta amino acids,
vi. D-configuration amino acid,
vii. non-proteinogenic amino acids, such as hydroxyproline (Hyp: L-hydroxyproline or (2S,4R)-4-Hydroxyproline), Octahydroindole-2-carboxylic acid (Oic), sarcosine (Sar), norleucine (Nle), α-aminoisobutyric acid (Aib), thienyl alanine (Thi), thiazolidine-4-carboxylic acid (Thz), phenylglycine (Phg), gamma aminobutyric acid (gaba), etc.
viii α-alkylated amino acids;

The compounds of the invention may comprise one or more modified amino acid or non-natural amino acid analogues in the Z^{a} peptide, the Z peptide or both the Z^{a} and Z peptides.

Suitably, at least one of the residues in the Z^{a} moiety is a modified or non-natural amino acid analogue.

The compounds of the invention may comprise multiple modified amino acid or non-natural amino acid analogues in the peptide sequence Z^{a}—Z.

In a preferred embodiment, Z^{a}—Z is a peptide of the formula:

SEQ ID NO: 145
S^{#}-D^{#}-S^{#}-K^{#}-N^{#}-T^{#}-A^{#}-L^{#}-W^{#}-F^{#}-G^{#}-P^{#}-R^{#}-L^{#}

wherein one or more of the residues in Z^{a} (i.e. “S^{#}”, “D^{#}”, “S^{#}”, “K^{#}”, “N^{#}”, “T^{#}”, “A^{#}”, “L^{#}”) or in Z (i.e. “W^{#}”, “F^{#}”, “G^{#}”, “P^{#}”, “R^{#}” or “L^{#}”) is modified or a non-natural amino acid analogue.

In another preferred embodiment, Z^{a}—Z is a peptide of the formula:

SEQ ID NO: 146
S^{#}-D^{#}-S^{#}-K^{#}-N^{#}-T^{#}-A^{#}-L^{#}-W-F-G-P-R-L

wherein one or more of the residues in Z^{a} (i.e. “S^{#}”, “D^{#}”, “S^{#}”, “K^{#}”, “N^{#}”, “T^{#}”, “A^{#}”, “L^{#}”) is modified or a non-natural amino acid analogue.

In a preferred embodiment, Z²—Z is a peptide of the formula:

SEQ ID NO: 147
L-W-F^{#}-G^{#}-P-R-L^{#}

the residues marked F^{#}, G^{#} and L^{#} are modified or non-natural amino acid analogues as described herein.

In a preferred embodiment Z^{a}—Z is a peptide of the formula:

SEQ ID NO: 148
L-W-[Phg]-[Sar]-PR-[n-me-L]

In certain embodiments, when R¹-L¹- is:

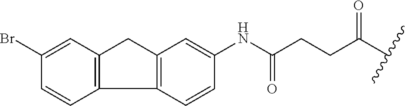 [SMILES: *C(=O)CCC(=O)Nc1ccc2c(c1)Cc1cc(Br)ccc1-2]

then at least one modified amino acid residue or non-natural amino acid residue in peptide Z^{a} or Z is not:
a beta amino acid; or
a D-configuration amino acid.

In certain embodiments, at least one modified amino acid residue or non-natural amino acid residue in peptide Z^{a} or Z is not:
a beta amino acid,
a D-configuration amino acid,
Hyp, Oic, Sar, Nle, orAib.

### Further Explanation of Compounds of the Invention

R¹, L¹ and R²

The terminal groups present at the N- and C-termini of the peptide backbone are designated R¹ and R² respectively. Thus R¹ is bonded (optionally via L¹) to the nitrogen atom of the N-terminal amino group of Z^{a} and R² is bonded to the C-terminal carbonyl carbon atom of Z.

In addition to comprising at least one modified or unnatural amino acid residue, the compounds of the invention may include further functionalisation, suitably at the N or C terminus. Suitably, the compounds may be functionalised to increase cuticle permeability or to increase stability. Suitably, the compound may be functionalised with an aromatic, aliphatic or lipophilic group. Suitably therefore, R¹ may be an aromatic, heteroaromatic, aliphatic or lipophilic group.

In certain embodiments, the compound may be functionalised with a lipophilic group such as a fatty acyl group. Fatty acyl groups include but are not limited to palmitoyl, butyryl, cerotoyl, decanoyl, docosenoyl, dodecanoyl, eleostearoyl, heptanoyl, hexanoyl, icosanoyl, icosenoyl, lignoceroyl, linoleoyl, lipoyl, myristoleoyl, nonanoyl, octadecanoyl, ocatanoyl, palmitoleoyl, stearoyl, undecanoyl, and valeryl. Suitably therefore the R¹ group may be palmitoyl ([Palm]), i.e.:

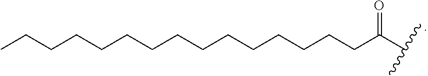 [SMILES: *C(=O)CCCCCCCCCCCCCCC]

In one embodiment, the compound may be functionalised with an aromatic group such as a benzyl or benzoyl group which may be a benzoic acid derivative or benzophenone derivative. Suitably therefore the R¹ group may be an aromatic group such as 4-benzoyl benzoic acid, or a derivative such as 4-benzoyl benzoyl.

In one embodiment, the compound may be functionalised with an acyl group. An “acyl” group is a group of the formula R^{3a}—C(O)— group wherein R^{3a} is a C₁₋₆alkyl, for example formyl, acetyl (Ac), propanoyl, butanoyl, or wherein R^{3a} is benzoyl. Suitably, the R^{3a} group may be R^{1b}—C(O)—, such as acetyl (Ac), i.e.:

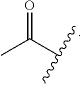 [SMILES: *C(C)=O]

In some embodiments, the R¹ group may be a biotin moiety. Thus, a biotin moiety may be incorporated into an amino acid residue (e.g. in a modified lysine side chain) or at the terminus.

In certain embodiments, the R¹ group may be substituted with a sugar moiety. An amino acid residue may also be modified with a sugar moiety, i.e. the amino acid residue may be a “sugar modified analogue”. The sugar moiety may be a monosaccharide or disaccharide. Examples of monosaccharides include glucose, 6-deoxyglucose, mannose, galactose, glucosamine, galactosamine, N-acetylglucosamine, N-acetylgalactosamine, glucuronic acid, allose, altrose, gulose, idose, fucose, talose, ribose, deoxyribose, arabinose, xylose, lyxose, ribulose, xylulose, fructose, psicose, sorbose or tagatose. Examples of disaccharides include sucrose, lactose, lactulose, allolactose, maltose, isomaltose, isomaltulose, trehalose, cellobiose, kojibiose, nigerose, sophorose, laminaribiose, gentiobiose, thiomaltose, mannobiose or their N—, C- or S-interglycosidic derivative. Most suitably, the sugar moiety is selected from glucosamine or galactosamine.

The sugar can be N-terminus modification or as part of Ser sidechain modification. As part of N-terminus, a spacer is usually added (e.g. succinic acid, Suc). An example of a R¹-L¹-Z^{a} sequence with such a modification is: sugar-Suc-SDSKNTAL (SEQ ID NO: 150). The notation “sugar” can be any of the sugars described herein, for example glucosamine or galactosamine.

Suitably the compound may also be modified with a (poly)alkyleneglycol polymer. This modification may be present in the R¹, L¹ or Z^{a} moiety. Suitably, the R¹ group may be a (poly)alkyleneglycol. The presence of the polymer may increase the ease of formulation of the compound. Preferred (poly)alkyleneglycols include polyethylene glycol (PEG). The compound may therefore be PEGylated, suitably by covalent attachment of polyethylene glycol to an amino acid residue in Z^{a}, at the R¹ position, or via a linker L¹. The PEG group may have any suitable terminal group, such as NH₂, OH or OMe. The linker L₁ may be a *—(C═O)—C₁₋₄alkylene group, where * denotes the point of attachment to Z.

PEG groups present as a linker portion or as part of a modified peptide chain have the formula [—(OCH₂CH₂)ₙ—], where n may preferably be an integer from 1 to 16.

R¹ is selected from hydrogen (which may be designated “H—” or “Hy-”), C₁₋₄ alkyl (e.g. methyl, ethyl, propyl, butyl), —N(R^{1a})—C(═N+(R^{1b})(R^{1c}))NR^{1d}R^{1c}, or —C(═N+(R^{1b})(R^{1c}))NR^{1d}R^{1e}; wherein each of R^{1a}, R^{1b}, R^{1c}, R^{1d} and R^{1e} is independently selected from hydrogen or C₁₋₄ alkyl (e.g. methyl, ethyl, propyl, butyl), preferably hydrogen or methyl.

In some embodiments, if R¹ is —C(═N+(R^{1b})(R^{1c}))NR^{1d}R^{1e}; each of R^{1a}, R^{1b}, R^{1c}, R^{1d} and R^{1e} are methyl, i.e. R¹ is —C(═N*Me₂)NMe₂.

When R¹=“H” (or “Hy”;), it typically indicates a free primary amino group at the N-terminus. The other hydrogen atom of the N-terminal amino group is typically invariant, regardless of the nature of R¹. Exceptionally, when the residue at the N-terminus is N-methylated, R¹ may still be indicated as H even though the N-terminal residue has a secondary amine group. Thus an N-methylated leucine residue at the N-terminus may be indicated as R¹-[n-me-L]- where R¹ is H. However, it could also be shown as simply R¹-L- where R¹ is methyl and the other hydrogen atom is not shown.

In some embodiments when R¹ is hydrogen, L¹ is absent or
*—(C═O)C₁₋₁₆-alkylene-NH— where * denotes the point of attachment to Z, e.g. R¹ is hydrogen and L¹ is *—(C═O)C₁₋₆-alkylene-NH—such as:

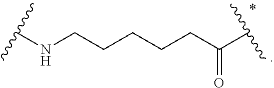 [SMILES: *NCCCCCC(*)=O]

In certain embodiments, L¹, if present, is *—(C═O)C₁₋₁₀-alkylene-NH— where * denotes the point of attachment to Z. For example, L¹ may be *—(C═O)Ce-alkylene-NH—, such as:

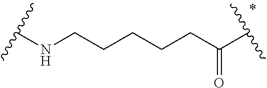 [SMILES: *NCCCCCC(*)=O]

which may be regarded as a residue of 6-aminohexanoic acid (Ahx).

In some embodiments when R¹ is hydrogen, L¹ is absent or *—(C═O)C₁₋₁₆-alkylene-NH— where * denotes the point of attachment to Z, e.g. R¹ is hydrogen and L¹ is *—(C═O)C₁₁alkylene-NH₂, where * denotes the point of attachment to Z, i.e.

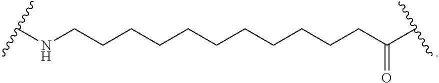 [SMILES: *NCCCCCCCCCCCC(*)=O]

In certain embodiments, L¹, if present, is *—(C═O)C₁₋₁₂-alkylene-NH— where * denotes the point of attachment to Z. For example, L¹ may be *—(C═O)C₆₋₁₂-alkylene-NH—, such as *—(C═O)C₁₁alkylene-NH₂, where * denotes the point of attachment to Z, i.e.

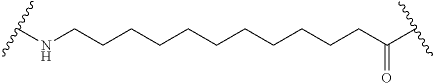 [SMILES: *NCCCCCCCCCCCC(*)=O]

which may be regarded as a residue of 12-aminododecanoic acid (Ado).

When L¹ is present, R¹ is typically hydrogen (H). For example, R¹ is hydrogen and L¹ is *—(C═O)C₁₋₆-alkylene-NH—such as:

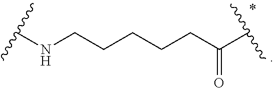 [SMILES: *NCCCCCC(*)=O]

In some embodiments, when R¹ is —C(═N⁺(R^{1b})(R^{1c}))NR^{1d}R^{1e}, L¹ is absent e.g. R¹ is —C(═N*Me₂)NMe₂ and L¹ is absent. In such embodiments the R¹ group forms the following guanidine based structure along with the N-terminal nitrogen (denoted “N—R” in the structure below):

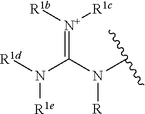

Typically, when L¹ is —(C═O)C₁₋₁₀-alkylene-NH—, R¹ is H.

Typically when R¹ is —C(═N⁺(R^{1b})(R^{1c}))NR^{1d}R^{1e}, L¹ is absent. When R¹ is —C(═N⁺(R^{1b})(R^{1c}))NR^{1d}R^{1e}″ and L¹ is absent, the R¹ group together with the N-terminal nitrogen of the peptide sequence Z form a guanidine based group (discussed above). Preferably “—C(═N⁺(R^{1b})(R^{1c}))NR^{1d}R^{1e}” is —C(═N*Me₂)NMe₂.

R² is NH₂, NR^{2a}H, NR^{2a}R^{2b}, or OR^{2a}; wherein each of R^{2a} and R^{2b} are as defined herein. Preferably R² is NH₂.

“Guanidyl” refers to the case where the R¹ is —C(═NMe₂)NMe₂ and the terminal structure formed by “Guanidyl-L-” is as follows:

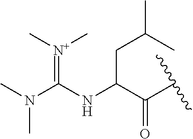 [SMILES: *C(=O)C(CC(C)C)NC(N(C)C)=[N+](C)C]

In one embodiment, the compound of the invention may be any of the CAPA-3 analogues listed herein. Examples of CAPA-3 analogue peptides include the compounds disclosed herein, and salts or solvates thereof, in particular:

| | |
|---|---|
| Hy-Ahx-SDSKNT[n-me-A]LWFGPRL-[NH₂] | SEQ ID 39 |
| Hy-SDSKNT[n-me-A]LWFGPRL-[NH₂] | SEQ ID 40 |
| Hy-SDSKNT[Peptoid-Phe]LWFGPRL-[NH₂] | SEQ ID 152 |
| Hy-SDSKNT[Peptoid-Ala]LWFGPRL-[NH₂] | SEQ ID 41 |
| Hy-SDSKNTA[Peptoid Leu]WFGPRL-[NH₂] | SEQ ID 42 |
| Hy-SDSKNTALW[Peptoid-Phe]GPRL-[NH₂] | SEQ ID 43 |
| Hy-SDSKNTA[n-me-L]WFGPRL-[NH₂] | SEQ ID 44 |
| Hy-SDSKNTALW[n-me-F]GPRL-[NH₂] | SEQ ID 45 |
| Hy-SDSKNTALWFGPR[n-me-L]-[NH₂] | SEQ ID 46 |
| Hy-SDSKNT[n-me-A]LWFGPR[n-me-L]- | SEQ ID 47 |
| [NH₂] | |
| [Peg8]-SDSKNT[n-me-A]LWFGPRL-[NH₂]; | SEQ ID 48 |
| [Palm]-SDSKNT[n-me-A]LWFGPRL-[NH₂]; | SEQ ID 49 |
| [ado]-SDSKNT[n-me-A]LWFGPRL-[NH₂]; | SEQ ID 50 |
| [2Abf-Suc]-SDSKNT[n-me-A]LWFGPRL- | SEQ ID 51 |
| [NH₂]; | |
| [Ind]-SDSKNT[n-me-A]LWFGPRL-[NH₂]; | SEQ ID 52 |
| [glucosamine-Suc]-SDSKNT[n-me- | SEQ ID 53 |
| A]LWFGPRL-[NH₂] | |
| [biotin]-SDSKNT[n-me-A]LWFGPRL-[NH₂] | SEQ ID 54 |
| [Biotin]-SDSKNTALWFGPRL-[NH₂] | SEQ ID 55 |
| Hy-SDS[Biotin-K]NTALWFGPRL-[NH₂] | SEQ ID 56 |
| Hy-S[Biotin-PEG-D]SKNTALWFGPRL-[NH₂] | SEQ ID 57 |
| Hy-SDS[Biotin-K]NT[n-me-A]LWFGPRL- | SEQ ID 58 |
| [NH₂] | |
| Hy-S[Biotin-PEG-D]SKNT[n-me- | SEQ ID 59 |
| ALWFGPRL-[NH₂] | |
| Hy-SDSKNT[a]LWFGPRL-[NH₂]; | SEQ ID 60 |
| Hy-SDSKNTA[l]WFGPRL-[NH₂]; | SEQ ID 61 |
| Hy-SDSKNTALW[f]GPRL-[NH₂]; | SEQ ID 62 |
| Hy-SDSKNTAL[w]FGPRL-[NH₂]; | SEQ ID 63 |
| Hy-SDSKNT[a][l]WFGPRL-[NH₂]; | SEQ ID 64 |
| Hy-SDSKNT[a]LW[f]GPRL-[NH₂]; | SEQ ID 65 |
| Hy-SDSKNTA[l]W[f]GPRL-[NH₂]; | SEQ ID 66 |
| Hy-SDSKNTA[a][l]W[f]GPRL-[NH₂]; | SEQ ID 67 |
| Hy-SDSKNT[a][l][w][f]GPRL-[NH₂]; | SEQ ID 68 |
| Hy-SDSKNT[B3A]LWFGPRL-[NH₂]; | SEQ ID 69 |
| Hy-SDSKNTA[B3L]WFGPRL-[NH₂]; | SEQ ID 70 |
| Hy-SDSKNTALW[B3F]GPRL-[NH₂]; | SEQ ID 71 |
| Hy-SDSKNT[Aib]LWFGPRL-[NH₂]; | SEQ ID 72 |
| Hy-SDSKNTALWFG[Hyp]RL-[NH₂]; | SEQ ID 73 |
| Hy-SDSKNT[Aib]LWFG[Hyp]RL-[NH₂]. | SEQ ID 74 |

A further example of a CAPA-3 analogue peptide, and a salt or solvate thereof, includes:

SEQ ID 75
Ac-LW-[Phg]-[Sar]-PR-[n-me-L]-[NH₂].
SEQ ID 151
Hy-SDSK[glucosamine-N]TALWFGPRL-[NH₂];

In some embodiments, the compounds of the invention may be in the form of a salt or solvate.

The compounds of the invention may be provided in combination with one or more additional active insecticides, such as those described herein.

### Activity

Suitably, the compounds have activity against hemipteran insects and/or dipteran insects. Preferably, the compounds have activity against hemipteran insects. The compounds of the invention may therefore find particular use against hemipteran insects.

The compounds typically increase insect mortality, for example when contacted topically to a suitable insect, or ingested by a suitable insect. Thus, the compounds described (and compositions containing them) may be regarded as insecticides, and may be referred to as “insect control agents”.

Without wishing to be bound by theory, any or all of the effects described may be mediated by agonist activity at the pyrokinin receptor of the target insects.

It is believed that, inter alia, the analogues described in this specification retain agonist activity while having superior stability compared to wild type CAPA-3 peptides, especially against proteases. Consequently, they are believed to have superior applicability as insecticides.

### Applications of the Present Invention

The invention provides a method of increasing insect mortality, comprising contacting an insect or insect population with a compound as described herein. The insect or insect population may be hemipteran, dipteran and/or lepidopteran insects.

The invention further provides a method of decreasing insect feeding, comprising contacting an insect or insect population with a compound as described herein. Suitably decreasing insect feeding on a plant or plant part. The insect or insect population may be hemipteran, dipteran and/or lepidopteran insects.

The compound may be applied directly to an insect or insect population. For example, it may be applied topically. Alternatively, the compound may be applied indirectly. For example, it may be applied to a substrate likely to come into contact with an insect or insect population. The substrate may be a plant or plant part, especially for Hemiptera or Diptera which represent pests of plants (whether crops or horticultural plants).

However, for insects which represent pests to humans, such as the Cimicidae family (e.g. bedbugs of the genus Cimex, such as Cimex lectularius) or the Reduviidae family (e.g. of the genus Rhodnius such as Rhodnius prolixus, or Triatoma such as Triatoma infestans) which can be vectors of human disease, the substrate may be a domestic surface or article, such as bedding, a mattress, or any other suitable domestic surface. The compound may be applied to the substrate in a form suitable for ingestion by an insect.

The invention further provides the use of a compound as described as a plant protection agent, and specifically for protecting a plant or plant part against hemipteran, dipteran and/or lepidopteran insects.

The invention further provides a method of inhibiting infestation of a plant or plant part by hemipteran, dipteran and/or lepidopteran insects comprising contacting the plant or plant part with a compound as described.

The method may be prophylactic. Thus, for example, the compound may be applied to the plant or plant part while the plant or part is free or substantially free of hemipteran, dipteran and/or lepidopteran insects.

Alternatively, the plant or plant part may already be colonised or infested by hemipteran, dipteran and/or lepidopteran insects. Thus, the invention further provides a method of reducing infestation of a plant or plant part, or of reducing hemipteran, dipteran and/or lepidopteran insect load on a plant or plant part, the method comprising contacting the plant or plant part with a compound as described.

In any of these embodiments, the compound may be provided as part of a composition, such as an insect control composition (e.g. insecticide composition) or a plant protection composition. Reference to application or use of a compound should therefore be construed as encompassing application or use of a suitable composition, unless the context demands otherwise.

The composition typically comprises a compound as described in combination with one or more ancillary component such as solvents, carriers, diluents, adjuvants, preservatives, dispersants, emulsifying agents, or synergists.

The composition may further comprise one or more additional active insecticides described herein.

The invention further provides a composition, e.g. an insect control composition or plant protection composition, comprising a compound of the invention in admixture with one or more solvents, carriers, diluents, adjuvants, preservatives, dispersants, emulsifying agents, or synergists. The composition may be an aqueous composition.

The invention includes the combination of the aspects and preferred features described except where such a combination is impermissible or expressly avoided.

### BRIEF DESCRIPTION OF THE FIGURES

FIG. 1 shows efficacies of endogenous CAPA-3 versus CAPA-3 analogues.

FIGS. 2 and 3 show efficacies of CAPA-3 analogues.

FIGS. 4 to 6 show efficacies of SB-P-15 in combination with CAPA-2 analogues, 2315, SB-P-9 and AH59

FIG. 7 shows results from an activation assay (IP1 assay) for M. persicae CAPA receptor stimulated by Drosophila CAPA peptide (positive control); SB-P-1 and SB-P-15.

FIG. 8 shows Scheme 1: Synthetic route taken toward SB-P-15. A general overview of the workflow involved is given in the inset.

FIG. 9 shows Scheme 2: Synthetic route taken toward SB-P-28.

FIG. 10 shows Scheme 3: Synthetic route taken toward SB-P-29. An overview of the typical workflow is given in the inset.

FIG. 11 shows Scheme 4: Synthetic route taken toward SB-P-30. An overview of the typical workflow is given in the inset.

FIG. 12 shows Scheme 5: Synthetic route taken toward SB-P-31.

FIGS. 13, 14 and 15 show further efficacy data for CAPA-3 peptides.

### DETAILED DESCRIPTION OF THE INVENTION

Aspects and embodiments of the present invention will now be discussed with reference to the accompanying figures. Further aspects and embodiments will be apparent to those skilled in the art. All documents mentioned in this text are incorporated herein by reference.

### Definitions

Throughout the present description and claims the conventional three-letter and one-letter codes for naturally occurring amino acids are used, i.e. A (Ala), G (Gly), L (Leu), I (Ile), V (Val), F (Phe), W (Trp), S (Ser), T (Thr), Y (Tyr), N (Asn), Q (Gln), D (Asp), E (Glu), K (Lys), R (Arg), H (His), M (Met), C (Cys) and P (Pro).

Generally, herein if the one letter code is accompanied by a “^{#}” symbol, it indicates that it may be either in its naturally occurring form, a modified form, or may be replaced with a non-proteinogenic amino acid. For Example, “A^{#}” encompasses unmodified alanine, a modified alanine (e.g. N-methyl alanine) or indicates that an alanine residue could be replaced by another non-natural amino acid analogue (e.g. α-aminoisobutyric acid (Aib)). Suitably, in the W^{#}—F^{#}-G^{#}-P^{#}—R^{#}-L^{#} motif, the residues may be in a naturally occurring form or a modified form. Suitably, in the W^{#}—F^{#}-G^{#}-P^{#}—R^{#}-L^{#} motif, a single residue may be replaced with a non-proteinogenic amino acid, e.g. WFG[Hyp]RL where proline is replaced by hydroxyproline.

‘Amino acid’ as referred to herein may refer to a naturally occurring amino acid or any other amino acid including synthetic amino acids, and non-proteinogenic amino acids. By “naturally occurring” in this context is meant the 20 amino acids encoded by the standard genetic code, sometimes referred to as proteinogenic amino acids.

The term amino acid is short for α-amino [alpha-amino]carboxylic acid. Each molecule contains a central carbon (C) atom, called the α-carbon, to which both an amino and a carboxyl group are attached. The remaining two bonds of the α-carbon atom are generally satisfied by a hydrogen (H) atom and the side chain, shown as R below:

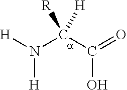

The modified amino acid in the present invention may be an α-alkylated amino acid, in which the α hydrogen atom has been replaced with a C₁₋₆alkyl group, e.g. a methyl or an ethyl group. Preferably, the α alkylated amino acid is an α-methylated amino acid, e.g. α-methyl-L-serine (given the notation [α-Me)S]):

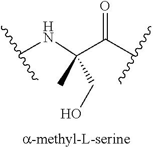 [SMILES: *N[C@@](C)(CO)C(*)=O]

Generally accepted three-letter codes and other abbreviations for other amino acids may also be employed, such as hydroxyproline (Hyp: L-hydroxyproline or (2S,4R)-4-Hydroxyproline), Octahydroindole-2-carboxylic acid (Oic), sarcosine (Sar), norleucine (NIe), α-aminoisobutyric acid (Aib), thiazolidine-4-carboxylic acid (Thz), phenylglycine (Phg) etc.

The term “Thz” indicates that an amino acid residue has been replaced with a thiazolidine-4-carboxylic acid residue, for example (R)-thiazolidine-4-carboxylic acid:

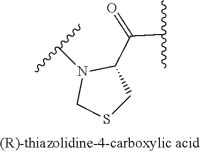 [SMILES: *C(=O)[C@@H]1CSCN1*]

Ahx indicates 6-aminohexanoic acid (also known as 6-aminocaproic acid or ε-aminocaproic acid). Ado indicates 12-aminododecanoic acid.

The notation “n-me” before an amino acid code is used to indicate an N-methylated amino acid residue. Thus, for example, “[n-me-V]” indicates N-methyl valine, “[n-me-A]” indicates N-methyl alanine and “[n-me-L]” indicates N-methyl leucine.

The residue sarcosine ([Sar]) is alternative name for an N-methyl glycine ([n-me-G]) residue:

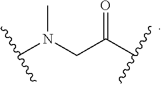 [SMILES: *C(=O)CN(*)C]

Such other amino acids may be shown in square brackets “[ ]” (e.g. “[Aib]”) when used in a general formula or sequence in the present specification, especially when the rest of the formula or sequence is shown using the single letter code.

Unless otherwise specified, amino acid residues in peptides of the invention are of the L-configuration. However, D-configuration amino acids may be incorporated as modifications. In the present context, an amino acid code written with a small letter may be used to represent the D-configuration of said amino acid, e.g. [a] represents the D-configuration of alanine.

Residues of beta amino acids may also be employed in compounds the invention. Such residues may be designated by a “β” symbol followed by the conventional code for the corresponding alpha amino acid. Thus, [βhL](or [B3L]) represents a residue of beta-homoleucine (3-amino-5-methylcaproic acid), [βA] represents a residue of beta-alanine (3-aminopropanoic acid), [(βhA](or [B3A]) represents a residue of beta-homoalanine, [βhV] represents a residue of beta-homovaline, sometimes referred to as beta-leucine (3-amino-4-methylpentanoic acid), [βhF](or [B3F]) represents a residue of beta-homo-phenylalanine, [βhP](or [B3P]) represents a residue of beta-homoproline.

A “peptoid analogue” of an amino acid is an analogue in which the side chain is connected to the nitrogen of the peptide backbone, instead of the α-carbon as in proteinogenic peptides. Such residues may be designated by a “Peptoid” notation followed by the conventional code for the corresponding alpha amino acid. An example of a peptoid includes a peptoid of phenylalanine:

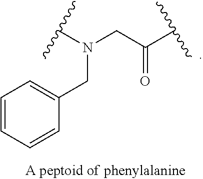 [SMILES: *C(=O)CN(*)Cc1ccccc1]

The above residue may also have the notation [Peptoid-F], [Peptoid-Phe] or [NPhe], indicating a peptoid of phenylalanine.

A “biotin modified” amino acid analogue is an amino acid residue which incorporates a biotin moiety as part of the side chain. For example, the notation “[Biotin-K]” indicates a lysine residue modified at the side chain to include a biotin moiety:

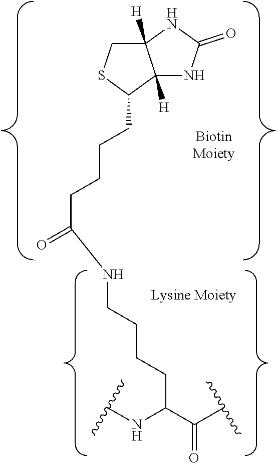

The biotin moiety may also be attached to the amino acid side chain via a linker group, e.g. a PEG or amino-terminated PEG group. For example, an aspartic acid (or glutamine) side chain modified with a PEG-linked biotin moiety:

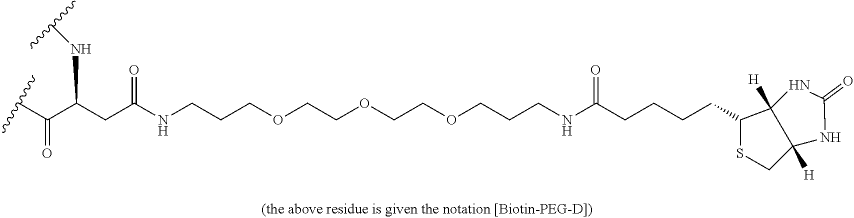

Alternatively, or in addition, a biotin moiety may be at a terminal position. Thus, R₁ may be biotin. The notation [Biotin]- represents a terminal biotin moiety, for example a partial sequence [Biotin]-S has the formula:

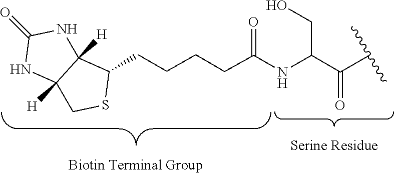 [SMILES: *C(=O)C(CO)NC(=O)CCCC[C@@H]1SC[C@@H]2NC(=O)N[C@@H]21]

The notation Cₓ₋ₓₓ refers to the number of carbon atoms in a functional group. The number in the ‘x’ positions is the lowest number of carbon atoms and the number in the ‘xx’ position denotes the highest number of carbon atoms. For example, C₁₋₆-alkyl refers to alkyl groups as defined herein having from 1 to 6 carbon atoms.

The notation i, n or t are used herein in relation to various alkyl groups in the normal way. Specifically, the suffixes refer to the arrangement of atoms and denote straight chain (‘n’) or branched (‘i’ or ‘t’) alkyl groups.

The term “alkyl” as used herein refers to a saturated linear or branched-chain monovalent hydrocarbon radical, wherein the alkyl radical may be optionally substituted. The number of carbon atoms in the alkyl group may be specified using the above notation, for example, when there are from 1 to 8 carbon atoms the term “C₁₋₈-alkyl” may be used. Examples of alkyl groups include methyl (Me, —CH₃), ethyl (Et, —CH₂CH₃), 1-propyl (n-Pr, n-propyl, —CH₂CH₂CH₃), 2-propyl (i-Pr, i-propyl, —CH(CH₃)₂), and 1-butyl (n-Bu, n-butyl, —CH₂CH₂CH₂CH₃).

An “alkylene,” “alkenylene,” or “alkynylene” group is an alkyl, alkenyl, or alkynyl group that is positioned between and serves to connect two other chemical groups. Thus, “C₁₋₆ alkylene” means a linear saturated divalent hydrocarbon radical of one to six carbon atoms or a branched saturated divalent hydrocarbon radical of three to six carbon atoms. Example alkylene groups include methylene (—CH₂—), 1,1-ethylene (—CH(CH₃)—), 1,2-ethylene (—CH₂CH₂—), 1,1-propylene (—CH(CH₂CH₃)—), and 2,2-propylene (—C(CH₃)₂—).

The term “alkenyl” as used herein refers to a linear or branched-chain monovalent hydrocarbon radical with at least one site of unsaturation, i.e., a carbon-carbon double bond. The alkenyl radical may be optionally substituted, and includes radicals having “cis” and “trans” orientations, or alternatively, “E” and “Z” orientations. The number of carbon atoms in the alkenyl group may be specified using the above notation, for example, when there are from 2 to 8 carbon atoms the term “C₂₋₈-alkenyl” may be used. Example alkenyl groups include, but are not limited to, ethenyl (—CH═CH₂), and prop-1-enyl (—CH═CHCH₃).

In the chemical structures drawn herein, the presence of “ [symbol] ” or “ [symbol] ” denotes a point of attachment or a radical for example, a radical as discussed in relation to various functional groups.

The term “aryl” means a cyclic or polycyclic aromatic ring having from 5 to 12 carbon atoms. The term aryl includes both monovalent species and divalent species. Aryl includes groups having a single ring and groups having more than one ring such a fused rings or spirocycles. In the case of groups having more than one ring, at least one of the rings is aromatic. The number of carbon atoms in the aryl group may be specified using the above notation, for example, when there are from 6 to 16 carbon atoms the term “C₆₋₁₆-aryl” may be used. Aryl groups may be optionally substituted. Examples of aryl groups include phenyl, naphthyl, biphenyl, phenanthrenyl, naphthacenyl, 1,2,3,4-tetrahydronaphthalenyl, 1H-indenyl, 2,3-dihydro-1H-indenyl, and fluorenyl. A preferred aryl group is fluorenyl.

The term 2Abf is known in the art and refers to 2-amino-7-bromofluorene, i.e.:

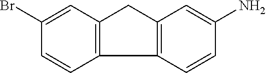 [SMILES: Nc1ccc2c(c1)Cc1cc(Br)ccc1-2]

The term “heteroaryl” or “heteroaromatic” means an aromatic mono-, bi-, or polycyclic ring incorporating one or more (for example 1-4, particularly 1, 2 or 3) heteroatoms selected from nitrogen, oxygen or sulfur. The term heteroaryl includes both monovalent species and divalent species. Examples of heteroaryl groups are monocyclic and bicyclic groups containing from five to twelve ring members, and more usually from five to ten ring members. The heteroaryl group can be, for example, a 5- or 6-membered monocyclic ring or a 9- or 10-membered bicyclic ring, for example a bicyclic structure formed from fused five and six membered rings or two fused six membered rings. Each ring may contain up to about four heteroatoms typically selected from nitrogen, sulfur and oxygen. Typically the heteroaryl ring will contain up to 3 heteroatoms, more usually up to 2, for example a single heteroatom. In one embodiment, the heteroaryl ring contains at least one ring nitrogen atom. The nitrogen atoms in the heteroaryl rings can be basic, as in the case of an imidazole or pyridine, or essentially non-basic as in the case of an indole or pyrrole nitrogen. In general the number of basic nitrogen atoms present in the heteroaryl group, including any amino group substituents of the ring, will be less than five.

Examples of heteroaryl include furyl, pyrrolyl, thienyl, oxazolyl, isoxazolyl, imidazolyl, pyrazolyl, thiazolyl, isothiazolyl, oxadiazolyl, thiadiazolyl, triazolyl, tetrazolyl, pyridyl, pyridazinyl, pyrimidinyl, pyrazinyl, 1,3,5-triazenyl, benzofuranyl, indolyl, isoindolyl, benzothienyl, benzoxazolyl, benzimidazolyl, benzothiazolyl, benzothiazolyl, indazolyl, purinyl, benzofurazanyl, quinolyl, isoquinolyl, quinazolinyl, quinoxalinyl, cinnolinyl, pteridinyl, naphthyridinyl, carbazolyl, phenazinyl, benzisoquinolinyl, pyridopyrazinyl, thieno[2,3-b]furanyl, 2H-furo[3,2-b]-pyranyl, 5H-pyrido[2,3-d]-o-oxazinyl, 1H-pyrazolo[4,3-d]-oxazolyl, 4H-imidazo[4,5-d]thiazolyl, pyrazino[2,3-d]pyridazinyl, imidazo[2,1-b]thiazolyl, imidazo[1,2-b][1,2,4]triazinyl. “Heteroaryl” also covers partially aromatic bi- or polycyclic ring systems wherein at least one ring is an aromatic ring and one or more of the other ring(s) is a non-aromatic, saturated or partially saturated ring, provided at least one ring contains one or more heteroatoms selected from nitrogen, oxygen or sulfur. Examples of partially aromatic heteroaryl groups include for example, tetrahydroisoquinolinyl, tetrahydroquinolinyl, 2-oxo-1,2,3,4-tetrahydroquinolinyl, dihydrobenzthienyl, dihydrobenzfuranyl, 2,3-dihydro-benzo[1,4]dioxinyl, benzo[1,3]dioxolyl, 2,2-dioxo-1,3-dihydro-2-benzothienyl, 4,5,6,7-tetrahydrobenzofuranyl, indolinyl, 1,2,3,4-tetrahydro-1,8-naphthyridinyl, 1,2,3,4-tetrahydropyrido[2,3-b]pyrazinyl and 3,4-dihydro-2H-pyrido[3,2-b][1,4]oxazinyl. A particularly preferred heteroaryl is indolyl, which may be joined to the peptide via a CH₂C(O) linker, and designated “[Ind]”.

Examples of five membered heteroaryl groups include but are not limited to pyrrolyl, furanyl, thienyl, imidazolyl, furazanyl, oxazolyl, oxadiazolyl, oxatriazolyl, isoxazolyl, thiazolyl, isothiazolyl, pyrazolyl, triazolyl and tetrazolyl groups.

Examples of six membered heteroaryl groups include but are not limited to pyridyl, pyrazinyl, pyridazinyl, pyrimidinyl and triazinyl.

A bicyclic heteroaryl group may be, for example, a group selected from:
a benzene ring fused to a 5- or 6-membered ring containing 1, 2 or 3 ring heteroatoms;
a pyridine ring fused to a 5- or 6-membered ring containing 1, 2 or 3 ring heteroatoms;
a pyrimidine ring fused to a 5- or 6-membered ring containing 1 or 2 ring heteroatoms;
a pyrrole ring fused to a 5- or 6-membered ring containing 1, 2 or 3 ring heteroatoms;
a pyrazole ring fused to a 5- or 6-membered ring containing 1 or 2 ring heteroatoms;
a pyrazine ring fused to a 5- or 6-membered ring containing 1 or 2 ring heteroatoms;
an imidazole ring fused to a 5- or 6-membered ring containing 1 or 2 ring heteroatoms;
an oxazole ring fused to a 5- or 6-membered ring containing 1 or 2 ring heteroatoms;
an isoxazole ring fused to a 5- or 6-membered ring containing 1 or 2 ring heteroatoms;
a thiazole ring fused to a 5- or 6-membered ring containing 1 or 2 ring heteroatoms;
an isothiazole ring fused to a 5- or 6-membered ring containing 1 or 2 ring heteroatoms;
a thiophene ring fused to a 5- or 6-membered ring containing 1, 2 or 3 ring heteroatoms;
a furan ring fused to a 5- or 6-membered ring containing 1, 2 or 3 ring heteroatoms; a cyclohexyl ring fused to a 5- or 6-membered heteroaromatic ring containing 1, 2 or 3 ring heteroatoms; and
a cyclopentyl ring fused to a 5- or 6-membered heteroaromatic ring containing 1, 2 or 3 ring heteroatoms.

Particular examples of bicyclic heteroaryl groups containing a six membered ring fused to a five membered ring include but are not limited to benzfuranyl, benzthiophenyl, benzimidazolyl, benzoxazolyl, benzisoxazolyl, benzthiazolyl, benzisothiazolyl, isobenzofuranyl, indolyl, isoindolyl, indolizinyl, indolinyl, isoindolinyl, purinyl (e.g., adeninyl, guaninyl), indazolyl, benzodioxolyl and pyrazolopyridinyl groups. A preferred bicyclic heteroaryl group is indolyl.

Particular examples of bicyclic heteroaryl groups containing two fused six membered rings include but are not limited to quinolinyl, isoquinolinyl, chromanyl, thiochromanyl, chromenyl, isochromenyl, chromanyl, isochromanyl, benzodioxanyl, quinolizinyl, benzoxazinyl, benzodiazinyl, pyridopyridinyl, quinoxalinyl, quinazolinyl, cinnolinyl, phthalazinyl, naphthyridinyl and pteridinyl groups.

The term halogen as used herein refers the one or more of fluorine (F), chlorine (Cl), bromine (Br) or iodine (I). The term “halo” or “halogeno” refers to fluoro, chloro, bromo and iodo.

The term “haloalkyl” refers to an alkyl group having on or more halogen substituent. The number of carbon atoms in the haloalkyl group may be specified using the above notation, for example, when there are from 1 to 8 carbon atoms the term “C-s-haloalkyl” may be used. Examples of haloalkyl groups include trifluoromethyl (—CF₃).

### Insect Control Agent

The term “insect control agent” refers to agents used to increase insect mortality (i.e. as insecticides). Thus an insect control agent may be administered to accelerate mortality of a given insect or insect population.

An increase in mortality used herein is intended to refer to an increase in the percentage of dead insects, as compared to the percentage of dead insects of an otherwise identical insect population which have not been exposed to the insect control agent of the invention.

Suitably, insect mortality may be calculated as number of dead insects/total number of insects per treated area. Suitably the treated area may be a well of a plate, or may be one or more leaves, or an entire plant.

An insect control agent may be used to reduce the size of an insect population, or inhibit growth of an insect population or inhibit feeding of an insect population (e.g. as compared to an otherwise identical insect population not exposed to the agent).

An insect control composition is a composition comprising an insect control agent as described.

### Plant Protection Agent

The term “plant protection agent” refers to agents when used to protect a plant or plant part against hemipteran, dipteran and/or lepidopteran insects, e.g. against infestation or colonisation, or being used as a food source by such insects (e.g. by the draining of sap). Infestation or colonisation may be by larvae (or nymphs), by adult insects, or by being used as a host or repository for eggs. The terms “infestation” and “colonisation” should not be construed as requiring the presence of the insects to be deleterious to the plant, however.

A plant protection agent may be applied inter alia for reducing insect load on a plant or plant part, for inhibiting (e.g. reducing the rate of) increase of insect load on a plant or plant part, or for maintaining a plant in an insect-free state, as compared to an otherwise identical plant having an insect population not exposed to the agent. Thus, the agent may be applied to a plant or plant part which already carries hemipteran, dipteran and/or lepidopteran insects, or to a plant or plant part which is free or substantially free of hemipteran, dipteran and/or lepidopteran insects.

A plant protection composition is a composition comprising a plant protection agent as described.

By ‘plant or plant part’, or ‘plant or part thereof’ referred to herein it is meant any part of a plant including but not limited to; the leaf, stem, root, flower, bud, bulb, and seed.

Suitable plants or parts thereof which may be protected by the agents of the present invention include crops and plants of agricultural, horticultural, or economic significance. Suitable plants may include any of the following or parts thereof: Musa textilis, Medicago sativa, Prunus dulcis, Pimpinella anisum, Malus sylvestris, Prunus armeniaca, Areca catechu, Arracacia xanthorhiza, Maranta arundinacea, Cynara scolymus, Helianthus tuberosus, Asparagus officinalis, Persea americona, Pennisetum americanum, Vigna subterranean, Musa paradisiaca, Hordeum vulgare, Phaseolus vulgaris, Phaseolus vigna spp., Beta vulgaris, Citrus bergamia, Rubus spp., Piper nigrum, Acacia mearnsii, Vaccinium spp., Bertholletia excelsa, Artocarpus altilis, Vicia faba, Brassica oleracea botrytis, Sorghum bicolor, Brassica oleracea gemmifera, Fagopyrum esculentum, Brassica oleracea capitate, Brassica rapa, Brassica spp., Theobroma cacao, Cucumis melo, Carum carvi, Elettaria cardamomum, Cynara cardunculus, Ceratonia siliqua, Daucus carota, Anacardium occidentale, Manihot esculenta, Ricinus communis, Brassica oleracea botrytis, Apium graveolens, Sechium edule, Prunus spp., Castanea sativa, Cicer arietinum, Cichorium intybus, Cichorium intybus, Capsicum spp., Cinnamomum verum, Cymbopogon nardus, Citrus medica, Citrus veticulata, Trifolium spp., Syzygium aromaticum, Cocos nucifera, Colocasia spp.; Xanthosoma spp., Coffee spp., Cola spp., Brassica napus, Zea mays, Valerianella locusta, Gossypium spp., Vigna unguiculate, Vaccinium spp., Lepidium sativum, Cucumis sativus, Ribes spp., Annona reticulata, Colocasia esculenta, Phoenix dactylifera, Moringa oleifera, Phaseolus spp., Allium sativum, Allium cepa, Pisum sativum, Triticum durum, Xanthosoma spp.; Colocasia spp., Solanum melongena, Cichorium endivia, Lygeum spartum, Foeniculum vulgare, Trigonella foenumgraecum, Ficus carica, Corylus avellane, Furcraea macrophylla, Linum usitatissimum, Phormium tenax, Pelargonium spp.; Geranium spp., Zingiber officinalis, Langenaria spp; Cucurbita spp., Cicer arietinum, Citrus paradise, Vitis vinifera, Lygeum spartum, Dactylis glomerata, Arachis hypogaea, Psidium guajava, Corylus avellane, Cannabis sativa, Crotalaria juncea, Agave fourcroydes, Lawsonia inermis, Humulus lupulus, Armoracia Rusticana, Indigofera tinctorial, Jasminum spp., Corchorus spp., Brassica oleracea acephala, Ceiba pentandra, Hibiscus cannabinus, Brassica oleracea gongylodes, Lavandula spp., Allium ampeloprasum, Citrus limon, Cymbopogon citratus, Lens culinans, Lespendeza spp., Lactuca sativa, Glycyrrhiza glabra, Citrus aurantifolia, Citrus limetta, Linum usitatissimum, Litchi chinensis, Eriobotrya japonica, Lupinus spp., Macadamia spp., Myristica fragrans, Agave atrovirens, Citrus reticulata, Mangifera indica, Manihot esculenta, Secale cereal, Mespilus germanica, Cucumis melo, Penicum miliaceum, Eleusine coracana, Setaria italica, Echinochloa crusgalli, Eleusine coracana; Mentha spp., Morus spp., Morus alba, Agaricus spp.; Pleurotus spp. Volvariella, Brassica nigra; Sinapis alba, Prunus persica, Phormium tenax, Guizotia abyssinica, Myristica fragrans, Avena spp., Elaeis guineensis, Abelmoschus esculentus, Olea europea, Papaver somniferum, Citrus sinensis, Citrus aurantium, Dactylis glomerate, Metroxylon spp., Borassus flabellifer, Carica papaya, Pastinaca sativa, Pyrus communis, Pisum sativum, Carya illinoensis, Capsicum annuum, Diospyros kaki; Diospyros virginiana, Cajanus cajan, Ananas comosus, Pistacia spp., Prunus domestica, Punica granatum, Citrus grandis, Solamum tuberosum, lpomoea batatas, Cucurbita spp., Chrysanthemum cineraraiefolium, Aspidosperma spp., Cydonia oblonga, Cinchona spp., Chenopodium quinoa, Raphanus sativus (including Cochlearia armoracia), Boehmeria nivea, Agrostis spp., Boehmeria nivea, Rheum spp., Oryza sativa; Oryza glaberrima, Rose spp., Hevea brasiliensis, Secale cereal, Lolium spp., Carthamus tinctorius, Metroxylon spp., Onobrychis viciifolia, Valerianellalocusta, Tragopogon porrifolius, Achras sapota, Citrus reticulata, Brassica ileracea capitate, Scorzonera hispanica, Sesamum indicum, Butyrospermum paradoxum, Agave sislana, Citrus aurantifolia, Glycine max, Triticum spelta, Spinacia oleracea, Secale cereal, Cucurbita spp., Fragaria spp., Sorghum bicolor Sudanense, Saccharum officinarum, Helianthus annuus, Crotalaria juncea, Citrus limetta, lopmoea batatas, Citrus reticulata, Xanthosoma sagittifolium, Manihot esculenta, Colocasia esculenta, Camellia sinensis, Eragrostis abyssinica, Phleum pratense, Nicotiana tabacum, Lycopersicum esculentum, Lotus spp., Aleurites spp., Brassica rapa, Urena lobate, Vanilla planifolia, Vicia sativa, Juglans spp., Citrullus lanatus, Acacia mearnsii, Triticum spp., Hordeum spp., Dioscorea spp., and Ilex paraguariensis.

Suitably, the plant or part thereof which may be protected by the agents of the present invention is selected from a plant which suffers from hemipteran, dipteran and/or lepidopteran insect infestations, or which attracts hemipteran, dipteran and/or lepidopteran insects. Suitably, the plant or part thereof which suffers from hemipteran, dipteran and/or lepidopteran insect infestations, or which attracts hemipteran, dipteran and/or lepidopteran insects is any of those listed above.

Suitably, the plant or part thereof which suffers from hemipteran insect infestations, or which attracts hemipteran insects, is any of those listed above.

Suitably, the plant or part thereof which suffers from dipteran insect infestations, or which attracts dipteran insects, is any of those listed above.

Suitably, the plant or part thereof which suffers from lepidopteran insect infestations, or which attracts lepidopteran insects, is any of those listed above.

In one embodiment, the plant is selected from a plant which suffers from or attracts hemipteran insect infestations, for example: cereal crops such as wheat (Triticum spp.), oats (Avena spp), rye (Secale spp.), barley (Hordeum spp.), rice (Oryza spp.) and corn (Zea spp.); fruit and vegetable crops including apples (Malus spp); pears (Pyrus spp); strawberry (Fragaria spp.), blueberry (Vaccinum spp.), blackberry (Rubus spp.), raspberry (Rubus spp.), citrus (Citrus spp.), olive (Olea spp.), durian (Durio spp.), longan (Dimocarpus spp.), litchi (L. chinensis), persimmon (Diospyros spp.); beans and peas (including but not limited to Phaseolus, Vigna, Pisum, Lens, Glycine, Cicer, Cajanus, Arachis spp), sugar beet (Beta vulgaris), sugar cane (Saccharum spp.), lettuce (Lactuca spp.), brassicas (Brassica spp.) including oil seed rape, alliums (Allium spp.), tomato (Solanum spp.), pepper (Capsicum spp.), asparagus (A. officinalis), melon, squash, pumpkins (Cucumis spp.), and tubers (potato) (Solanum spp.), or a part thereof.

In one embodiment, the plant is selected from a plant which suffers from or attracts aphid insect infestations, suitably M. persicae insect infestations, including Solanaceae, Cruciferae, and Leguminosae for example: cereal crops such as wheat (Triticum spp including winter wheat Triticum aestivum L); fruit and vegetable crops including peach (Prunus spp.), strawberry (Fragaria spp.), blueberry (Vaccinum spp.), blackberry (Rubus spp.), raspberry (Rubus spp.), brassicas (Brassica spp.) such as oil seed rape, lettuce (Lactuca spp.), tomato (Solanum spp.), pepper (Capsicum spp.), beans and peas (including but not limited to Vigna, Pisum spp), melon, squash, pumpkins (Cucumis spp.), citrus (Citrus spp.), and tubers (potato) (Solanum spp.), or a part thereof. In one embodiment, the plant is a vegetable crop, suitably a brassica spp.

In one embodiment, the plant is selected from a plant which suffers from or attracts dipteran insect infestations, for example: cereals (Triticum spp.); oats (Avena spp);, rye (Secale spp.); barley (Hordeum spp,) rice (Oryza spp.) and corn (Zea spp.); beans and peas (including but not limited to Phaseolus, Vigna, Pisum, Lens, Glycine, Cicer, Cajanus, Arachis spp); fruit crops including apples (Malus spp), pears (Pyrus spp), strawberry (Fragaria spp.), blueberry (Vaccinum spp.), blackberry (Rubus spp.), raspberry (Rubus spp.), cherry, plum, apricot, peach, nectarine (Prunus spp.), blackcurrant, redcurrant, whitecurrant, gooseberry (Ribes spp.), kiwi fruit (Actinidia spp), papaya (Carica spp.), avocado (Persea spp.), mango (Mangifera indica L), longan (Dimocarpus spp.), litchi (L. chinensis), grapes (Vitis spp.), fig (Ficus spp.), passionfruit (Passiflora spp.), Asian pears (Pyrus spp), citrus (Citrus spp.), and olive (Olea spp.); vegetable crops including alliums (Allium spp.), aubergine, tomato (Solanum spp.) and peppers (Capsicum spp.), lettuce (Lactuca spp.), brassicas (Brassica spp.) and courgette, melon, squash, pumpkins (Cucumis spp.); Apiaceae root crops including carrot (Daucus spp.), parsnip (Pastinaca spp.), or a part thereof.

In one embodiment, the plant is selected from a plant which suffers from or attracts lepidoptera insect infestations, for example: cereal crops such as wheat (Triticum spp.), oats (Avena spp), rye (Secale spp.), barley (Hordeum spp.), rice (Oryza spp.) and corn (Zea spp.); fruit and vegetable crops including apples (Malus spp); pears (Pyrus spp); tree nuts (including for example almonds (P. amygdalus), pistachio (Pistacia vera), walnuts (Juglandaceae), hazlenuts (Corylus)); avocado, including Persea Americana (Lauraceaea), blueberry (Vaccinum spp.), citrus (Citrus spp.), olive (Olea spp.), durian (Durio spp.), longan (Dimocarpus spp.), litchi (L. chinensis), persimmon (Diospyros spp.); beans and peas (including but not limited to Phaseolus, Vigna, Pisum, Lens, Glycine, Cicer, Cajanus, Arachis spp), sugar beet (Beta vulgaris), sugar cane (Saccharum spp.), lettuce (Lactuca spp.), brassicas (Brassica spp.) including oil seed rape, alliums (Allium spp.), tomato (Solanum spp.), pepper (Capsicum spp.), asparagus (A. officinalis), melon, squash, pumpkins (Cucumis spp.), and tubers (potato) (Solanum spp.), or a part thereof.

### Hemipteran Insects

The compounds and compositions of the invention suitably have activity against insects of the Order Hemiptera, which comprises groups including aphids, planthoppers, leafhoppers, stink bugs, shield bugs and cicadas.

Hemipterans are defined by distinctive mouthparts in the form of a “beak”, comprising modified mandibles and maxillae which form a “stylet”, sheathed within a modified labium.

Many insects within these groups have endogenous neuropeptides with sequence homology to the peptides described herein, suggesting that these analogues may have activity against those insects.

The insects may belong to the sub-order Sternorrhyncha, e.g. to the super-family of Aphidoidea (aphid superfamily), Aleyrodoidea (whiteflies), Coccoidea (scale insects), Phylloxeroidea (including Phylloxeridae or “phylloxerans”, and Adelgidae or woolly conifer aphids) or Psylloidea (jumping plant lice etc.).

Thus, the insects may be aphids, i.e. members of the aphid superfamily (Aphidoidea). Aphids (Hemiptera: Aphididae) are one of the most significant groups of agricultural pests and are vectors in the transmission of approximately 50% of all insect transmitted plant viruses. Within that superfamily, the aphids may be part of the family Aphididae, which contains sub-families Aiceoninae, Anoecilnae, Aphidinae, Baltichaitophorinae, Calaphidinae, Chaitophorinae, Drepanosiphinae, Eriosomatinae, Greenideinae, Hormaphidinae, Israelaphidinae, Lachninae, Lizeriinae, Macropodaphidinae, Mindarinae, Neophyllaphidinae, Phloeomyzinae, Phyllaphidinae, Pterastheniinae, Saltusaphidinae, Spicaphidinae, Taiwanaphidinae, Tamaliinae and Thelaxinae.

The aphids may, for example, be of the genus Acyrthosiphon (e.g. Acyrthosiphon pisum), Aphis (e.g. Aphis gossypii, Aphis glycines), Diuraphis (e.g. Diuraphis noxia) Macrosiphum (e.g. Macrosiphum rosae, Macrosiphum euphorbiae), Myzus (e.g. Myzus persicae), Rhopalosiphum (e.g. Rhopalosiphum padi) or Sitobion (e.g. Sitobion avenae).

Myzus persicae (peach potato aphid) is the most economically important aphid crop pest worldwide, with a global distribution and host range encompassing more than 400 species in 40 different plant families. For example, it is a major pest of agricultural crops including fruit and potatoes, and act as a vector for viruses.

Macrosiphum rosae, (rose aphid) is an important horticultural pest, especially of cultivated species of Rosa, and is a vector in the transmission of 12 plant viruses including the strawberry mild yellow edge virus.

Aphis gossypii (cotton or melon aphid) is a pest of Curcibitae and cotton.

Other than aphids, the insects may, for example, be of the Adelgidae family, e.g. of the genus Adelges (e.g. Adelges tsugae).

The insects may be of the Aleyrodidae family, e.g. of the genus Bemisia (e.g. Bemisia tabaci) or Trialeurodes (e.g. Trialeurodes vaporariorum).

The insects may be of the Psylloidea family, e.g. of the genus Pachypsylla (e.g. Pachypsylla venusta).

As examples of hemipteran insects outside the sub-order Sternorryncha, the insects may be of the Cimicidae family, e.g. of the genus Cimex (bed bugs), e.g. Cimex lectularius.

The insects may be of the Cicadellidae family, e.g. of the genus Cuerna (e.g. Cuerna arida), Graminella (e.g. Graminella nigrifrons) or Homalodisca (e.g. Homalodisca vitripennis).

The insects may be part of the Delphacidae family, e.g. of the genus Nilaparvata (e.g. Nilaparvata lugens) or Sogatella (e.g. Sogatella furcifera). For example, Nilaparvata lugens (brown planthopper) is a pest of rice crops, especially in Asia.

The insects may be of the Liviidae family, e.g. of the genus Diaphorina (e.g. Diaphorina citri).

The insects may be part of the Miridae family, e.g. of the genus Pseudatomoscelis (e.g. Pseudatomoscelis seriatus), Lygus (e.g. Lygus hesperus) or Tupiocoris (e.g. Tupiocoris notatus). For example, Pseudatomoscelis seriatus (cotton fleahopper) is a pest of cotton.

The insects may be of the Pentatomidae family, e.g. of the genus Acrosternum (e.g. Acrosternum hilare), Banasa (e.g. Banasa dimiata), Euschistus (e.g. Euschistus servus, Euschistus heroes), Halyomorpha (e.g. Halyomorpha halys), Murgantia (e.g. Murgantia histrionica), Nezara (e.g. Nezara viridula), Plautia (e.g. Plautia stali), or Podisus (e.g. Podisus maculiventris). For example, Acrosternum hilare (green stink bug) is a significant pest of cotton. Euschistus servus (brown stink bug) is a pest of many agricultural crops including seeds, grains, nuts and fruits, especially in the southern USA. Nezara viridula is a pest of grain and soybean crops, especially in Brazil.

The insects may be of the Pyrrhocoridae family, e.g. of the genus Pyrrhocoris (e.g. Pyrrhocoris apterus).

The insects may be of the Reduviidae family, e.g. of the genus Rhodnius (e.g. Rhodnius prolixus), or Triatoma (e.g. Triatoma infestans). Rhodnius prolixus is a vector of human disease (Chagas disease).

The insects may be of the Triozidae family, e.g. of the genus Acanthocasuarina (e.g. Acanthocasuarina muellerianae).

In one embodiment, the insect may be selected from the following species: H. halys, E. heroes, A. hilare, A. gossypii, E. servus, M. persicae, N. viridula, N. lugens, P. seriatus, and R. prolixus.

In one embodiment, the insect is of the species M. persicae.

### Dipteran Insects

The compounds and compositions of the invention may have activity against insects of the Order Diptera,

In particular, they may have activity against insects of the family Drosophilidae, such as fruit flies, including those of genus Drosophila, such as Drosophila suzukii. They may also have activity against insects of the family Tephritidae, including those of the genera Anastrepha (Anastrepha spp.); Bactrocera (Bactrocera spp.); Ceratitis (Ceratitis spp.); Dacus (Dacus spp.); Rhagoletis (Rhagoletis spp.); Tephritis (Tephritis spp.).

The families Drosophilidae and Tephritidae together are commonly referred to as fruit flies.

The compounds may also have activity against other important dipteran pests, such as flies of the family Chloropidae (chloropid flies) and those of the genera:
Phytomyza (e.g. Phytomyza angelicastri);
Melani (e.g. Melani agromyza);
Antherigona (e.g. Antherigona spp);
Delia (e.g. Delia radicum);
Contarinia (e.g. Contarinia sorghicola):

For more detail on these, and other examples, see Developing the Arsenal Against Pest and Vector Dipterans: Inputs of Transgenic and Paratransgenic Biotechnologies, Ogaugwu and Durvasula, IntechOpen, 2017: DOI:10.5772/66440

### Lepidopteran Insects

The compounds and compositions of the invention may have activity against insects of the Order Lepidoptera.

In particular, they may have activity against insects Heliothis peltigera, H. virescens, Plutella xylostella, Spodoptera spp., and Cydia pomonella (Codling Moth), Larvae of Heliothis spp., including peltigera and virescens Spodoptera littoralis (which represent a large variety of Heliothinae and Spodoptera moth species and are world-wide agricultural pests), Plutella xylostella (diamondback moth, most important world-wide pest of Brassicas).

### Methods and Uses of the Invention

The present invention provides the use of a compound as described herein as an insect control agent, specifically in methods of increasing mortality in insects that encode the CAPA-3 peptide, or a method of inhibiting infestation of a plant by insects that encode the CAPA-3 peptide.

The present invention also provides the use of a compound as described herein as an insect control agent, specifically in methods of increasing hemipteran, dipteran and/or lepidopteran insect mortality, or a method of inhibiting infestation of a plant by hemipteran, dipteran and/or lepidopteran insects. Preferably, the present invention relates to the use of a compound as described herein as an insect control agent, specifically in methods of increasing hemipteran insect mortality, or a method of inhibiting infestation of a plant by hemipteran insects.

Suitably, the compound may be for use as an insect control agent wherein the insect is of the order hemipteran.

Suitably, the compound may be for use as an insect control agent wherein the insect is of the genus Drosophila.

Suitably, the compound may be for use as an insect control agent wherein the insect is Drosophila suzukii.

In one embodiment, there is provided a method of increasing dipteran insect mortality, comprising contacting a dipteran insect or dipteran insect population with a compound or composition of the invention.

In one embodiment, there is provided a method of inhibiting infestation of a plant by dipteran insects comprising contacting the plant with a compound or composition of the invention.

In some embodiments, the compound for use against insects of the order diptera is any one of the specific compounds described herein.

In one particular embodiment, there is provided a method of increasing Drosophila suzukii mortality, comprising contacting a Drosophila suzukii insect or insect population with a compound or composition of the invention.

In one particular embodiment, there is provided a method of inhibiting infestation of a plant by Drosophila suzukii comprising contacting the plant with compound AH382.

Suitably contacting may comprise feeding or spraying, for example. Suitably feeding may be encouraged via bait attractants, which may be comprised in a composition of the invention, as explained below.

Suitably, the compound or composition as defined herein may be for use as an insect control agent wherein the insect is of the order hemipteran. Suitably, the compound or composition as defined herein may be for use as an insect control agent wherein the insect is of the genus Myzus. Suitably, the compound or composition as defined herein may be for use as an insect control agent wherein the insect is Myzus persicae.

In one embodiment, there is provided a method of increasing hemipteran insect mortality, comprising contacting a hemipteran insect or hemipteran insect population with a compound or composition as defined herein.

In one embodiment, there is provided a method of inhibiting infestation of a plant by hemipteran insects comprising contacting the plant with a compound or composition as defined herein.

In one particular embodiment, there is provided a method of increasing Myzus persicae mortality, comprising contacting a Myzus persicae insect or insect population with a compound or composition as defined herein.

In one embodiment, there is provided a method of inhibiting infestation of a plant by Myzus persicae comprising contacting the plant with a compound or composition as defined herein.

In one particular embodiment, there is provided a method of increasing Myzus persicae mortality, comprising contacting a Myzus persicae insect or insect population with compound or composition as defined herein.

In one particular embodiment, there is provided a method of inhibiting infestation of a plant by Myzus persicae comprising contacting the plant with compound or composition as defined herein.

Suitably, the compound or composition as defined herein may be for use as an insect control agent wherein the insect is of the order hemipteran. Suitably, the compound or composition as defined herein may be for use as an insect control agent wherein the insect is of the genus Rhopalosiphum. Suitably, the compound or composition as defined herein may be for use as an insect control agent wherein the insect is Rhopalosiphum padi.

In one embodiment, there is provided a method of increasing hemipteran insect mortality, comprising contacting a hemipteran insect or hemipteran insect population with a compound or composition as defined herein.

In one embodiment, there is provided a method of inhibiting infestation of a plant by hemipteran insects comprising contacting the plant with a compound or composition as defined herein.

In one particular embodiment, there is provided a method of increasing Rhopalosiphum padi mortality, comprising contacting a Rhopalosiphum padi insect or insect population with a compound or composition as defined herein.

In one embodiment, there is provided a method of inhibiting infestation of a plant by Rhopalosiphum padi comprising contacting the plant with a compound or composition as defined herein.

In one particular embodiment, there is provided a method of increasing Rhopalosiphum padi mortality, comprising contacting a Rhopalosiphum padi insect or insect population with compound or composition as defined herein.

In one particular embodiment, there is provided a method of inhibiting infestation of a plant by Rhopalosiphum padi comprising contacting the plant with compound or composition as defined herein.

Suitably, the compound or composition as defined herein may be for use as an insect control agent wherein the insect is of the order lepidopteran. Suitably, the compound or composition as defined herein may be for use as an insect control agent wherein the insect is of the genus Plutella. Suitably, the compound or composition as defined herein may be for use as an insect control agent wherein the insect is Plutella xylostella.

In one embodiment, there is provided a method of increasing hemipteran insect mortality, comprising contacting a hemipteran insect or hemipteran insect population with a compound or composition as defined herein.

In one embodiment, there is provided a method of inhibiting infestation of a plant by hemipteran insects comprising contacting the plant with a compound or composition as defined herein.

In one particular embodiment, there is provided a method of increasing Plutella xylostella mortality, comprising contacting a Plutella xylostella insect or insect population with a compound or composition as defined herein.

In one embodiment, there is provided a method of inhibiting infestation of a plant by Plutella xylostella comprising contacting the plant with a compound or composition as defined herein.

In one particular embodiment, there is provided a method of increasing Plutella xylostella mortality, comprising contacting a Plutella xylostella insect or insect population with compound or composition as defined herein.

In one particular embodiment, there is provided a method of inhibiting infestation of a plant by Plutella xylostella comprising contacting the plant with compound or composition as defined herein.

Suitably contacting may comprise feeding or spraying, for example. In some embodiments, when the contacting is by feeding.

Suitably the compound may be contacted with the insect or insect population, or plant or plant part, at any suitable concentration which is effective. Suitably the concentration of the compound is between 10⁻³ to 10⁻⁹ M, suitably between 10⁻⁴ to 10⁻⁶ M, suitably between 10⁻⁴ to 10⁻⁵M.

The compound of the invention may be used in combination with a further insecticide, as described herein. Suitably, the insecticide is a further CAPA analogue.

The methods of the invention may further comprise contacting the insect population or plant with a further insecticide agent, such as those described herein. Suitably, the insecticide is a further CAPA analogue.

### Beneficial Insect Species

The compounds and compositions of the invention may be substantially non-toxic to beneficial insect species, including species which prey on pests and pollinator species. Important pollinator species, such as insects of the superfamily Apoidea, including bees, such as the Apidae, e.g. those of the genus Bombus, such as Bombus terrestris. Important predatory species include Coccinellidae (lady bugs) such as Adalia bipunctata.

By substantially non-toxic, it is meant that the compounds and compositions of the invention do not cause death of the beneficial insect species (e.g. pollinator species), suitably that they do not cause premature death of the beneficial insect species (e.g. pollinator species). It is also meant that the compounds and compositions of the invention do not cause any detrimental side effects to the beneficial insect species (e.g. pollinator species), for example they do not have a negative effect on feeding behaviour, or ability to move.

### Compositions

Compositions of the invention, or for use in accordance with the invention, typically comprise a compound as described in combination with one or more ancillary component such as solvents, carriers, diluents, adjuvants, preservatives, dispersants, emulsifying agents, or synergists.

Suitably, the compositions of the invention are aqueous compositions.

The compound content of the composition can vary within wide limits. The compound concentration of the composition can be from 0.0000001 to 95% by weight of the compound, preferably between 0.0001 and 1% by weight.

The compositions of the invention, or for use in accordance with the invention, may comprise more than one compound of the invention in combination. Therefore the compositions of the invention may comprise a first compound of the invention and a second compound of the invention. Suitably the first and second compound may be any of those described herein, and may be present in the composition in any relative proportion.

The composition may be an aqueous composition, e.g. a saline composition. The aqueous composition may contain one or more buffers, such as a phosphate buffer (e.g. phosphate buffered saline) or a Tris buffer. Alternatively the composition may be an oil dispersion or an emulsion, e.g. an oil and water emulsion. Alternatively the composition may be a suspension, powder, foam, paste, granule, aerosol, impregnated natural and synthetic substance, or encapsulated in polymeric substance for example. A suitable form of the composition may be chosen for the intended use having regard to the target insect, and to its habitat.

Adjuvants may enhance product performance, for example, by increasing the efficiency of the delivery of active ingredients, reducing the level of active ingredient required, or extending the spectrum of effectiveness.

Different types of adjuvants offer various benefits and advantages, which are achieved by modulating properties such as spray formation, spray retention, wetting, deposit formation or uptake.

Adjuvants modulating spray formation may influence spray quality by reducing spray drift and wastage, allowing more of the product to reach the target. This can reduce use rates, leading to a better environmental profile and a potentially more cost effective solution. Such adjuvants include non-ionic surfactants and emulsifier blends.

Adjuvants modulating spray retention may dissipate the kinetic energy of the droplet during impact, meaning the likelihood of bounce or run-off is reduced. Such adjuvants include alkyl polyglucosides, alkoxylated alcohols, and polyoxyethylene monobranched alcohols (e.g. polyoxyethylene (8) monobranched alcohol).

Adjuvants modulating wetting properties (i.e. wetting agents) may reduce surface tension and contact angle, leading to enhanced coverage. Such adjuvants include polyoxyethylene sorbitan monolaurate (e.g. polyoxyethylene (8) sorbitan monolaurate), surfactant blends, and alkyl polyglucosides.

Adjuvants modulating deposit formation may influence evaporation of water from the droplet and thus provide a more homogeneous distribution. Such adjuvants include alkoxylated polyol esters, polyoxyethylene sorbitan monolaurate (e.g. polyoxyethylene (12) sorbitan monolaurate), and alkyl polyglucoside.

Adjuvants modulating uptake can improve penetration and uptake of active ingredients. e.g. through the insect cuticle, resulting in increased bioavailability. Such adjuvants include alkoxylated polyol esters and polyoxyethylene sorbitan monolaurate (e.g. polyoxyethylene (12) sorbitan monolaurate and polyoxyethylene (16) sorbitan monolaurate).

Dispersants may be aqueous or non-aqueous. An oil dispersion (OD) formulation typically comprises a solid active ingredient dispersed in oil. The oil can vary from paraffinic to aromatic solvent types and vegetable oil or methylated seed oils. Typically the active ingredient is uniformly suspended in the oil phase. Although primarily used for water sensitive active ingredients, OD formulations have extended to other active ingredients due to their better spray retention, spreading, foliar uptake, and penetration enhancement (e.g. across the insect cuticle) as the carrier oil often acts as an adjuvant.

Oils suitable for use in OD dispersions include linseed, rapeseed and soyabean oils.

Aqueous dispersants may be used, for example, to improve stability in the spray tank after dilution in water, and may include modified styrene acrylic polymers, and polymeric amphoteric dispersants and adjuvants.

An emulsifier may be employed to emulsify a continuous oil phase into water when an OD formulation is diluted prior to being sprayed. The emulsifier may be selected based upon its ability to spontaneously form the emulsion. Their performance is primarily dictated by the nature of the surfactant and their collective effect on how they arrange themselves at the oil/water interface. Examples include polyoxyethylene sorbitol hexaoleate (e.g. polyoxyethylene (40) sorbitol hexaoleate), emulsifier blends, and calcium alkylaryl sulphonate.

The composition may further comprise an adhesive or a dye.

The compound may be provided in the form of a concentrate, for dilution prior to application. Alternatively the compound may be provided in a solid form to be suspended or dissolved prior to formulation.

The composition may be a bait composition for ingestion by the target insect.

A bait composition may comprise one or more phagostimulants, i.e. a substance which will entice the insect to ingest the compound. Phagostimulants may include artificial sweeteners, amino acids, other peptides or proteins and carbohydrates (e.g. glucose, fructose, sucrose, maltose) etc. Examples include honey, syrups and aqueous solutions of sucrose.

Commercially available base formulations may also be suitable for use in formulating the compounds described in this specification, such as Armid® FMPC (Akzo Nobel).

The composition may comprise one or more synergists, i.e. compounds which increase the efficacy of insecticides against their targets, often by inhibiting an insect's ability to metabolise the active agent. Common synergists include piperonyl butoxide and MGK-264 (n-octyl bicycloheptane dicarboximide), or peptidase inhibitors.

There is also provided a combination comprising a compound of the invention, or a salt or solvate thereof, in combination with one or more additional active insecticides. The additional active insecticide may be selected from pyrethrins or pyrethroids, or other peptide analogues. The insecticides may also include, for example, phosphates, carbamates, carboxylates, chlorinated hydrocarbons, phenylureas and substances produced by microorganisms. The additional active insecticide may be a CAPA-1 or CAPA-2 analogue, preferably, a CAPA-2 analogue.

The composition may further comprise one or more additional active insecticides. The compositions of the invention, or for use in accordance with the invention, may comprise a compound of the invention in combination with a further CAPA peptide, e.g. a CAPA-1 or CAPA-2 analogue. Suitably, the compositions of the invention may comprise a CAPA-2 analogue. The CAPA analogue may be selected from 2315, SB-P-9 and AH59, which have the sequences below:

[2315]-
SEQ ID 115
Hy-ASG[βhL]VAFPRV-NH₂;
[SB-P-9]-
SEQ ID 76
Hy-[Peg8]-ASGL[βhL]AFPRV-NH₂;
[AH59]-
SEQ ID -77
Palmitoyl-LVAFPRV-NH₂.

The choice of ancillary or additional insecticides will typically depend on the particular target species. The composition may further comprise one or more additional, attractants, sterilizing agents, acaricides, nematicides, fungicides, growth-regulating substances or herbicides.

### REFERENCES

A number of publications are cited in order to more fully describe and disclose the invention and the state of the art to which the invention pertains. Full citations for these references are provided below. The entirety of each of these references is incorporated herein by reference.
Koyama T, Terhzaz S, Naseem M T, Nagy S, Rewitz K, Dow J A T, Davies S A, Halberg K V. A nutrient-responsive hormonal circuit mediates an inter-tissue program regulating metabolic homeostasis in adult Drosophila. Nat Commun. 2021
Kean L, Cazenave W, Costes L, Broderick K E, Graham S, Pollock V P, Davies S A, Veenstra J A, Dow J A. Two nitridergic peptides are encoded by the gene capability in Drosophila melanogaster. Am J Physiol Regul Integr Comp Physiol. 2002
Yeoh J G C, Pandit A A, Zandawala M, Nassel D R, Davies S A, Dow J A T. DINeR: Database for Insect Neuropeptide Research. Insect Biochem Mol Biol. 2017
Ahn S J, Corcoran J A, Vander Meer R K, Choi M Y. Identification and Characterization of GPCRs for Pyrokinin and CAPA Peptides in the Brown Marmorated Stink Bug, Halyomorpha halys (Hemiptera: Pentatomidae). Front Physiol. 2020
Audsley N and Down R E, G protein coupled receptors as targets for next generation pesticides. Insect Biochem Molec 67: 27-37 (2015).
Halberg K A, Terhzaz S, Cabrero P, Davies S A and Dow J A T, Tracing the evolutionary origins of insect renal function. Nat Commun 6 (2015).
Dow J A, Insights into the Malpighian tubule from functional genomics. J Exp Biol 212: 435-445 (2009).
Huesmann G R, Cheung C C, Loi P K, Lee T D, Swiderek K M and Tublitz N J, Amino acid sequence of CAP2b, an insect cardioacceleratory peptide from the tobacco hawkmoth Manduca sexta. FEBS Lett 371: 311-314 (1995).
Davies S A, Cabrero P, Povsic M, Johnston N R, Terhzaz S and Dow J A T, Signaling by drosophila capa neuropeptides. Gen Comp Endocr 188: 60-66 (2013).
Terhzaz S, Teets N M, Cabrero P, Henderson L, Ritchie M G, Nachman R J, Dow J A T, Denlinger D L and Davies S A, Insect capa neuropeptides impact desiccation and cold tolerance. Proc Natl Acad Sci 201501518 (2015).
Terhzaz S, Alford L, Yeoh J G C, Marley R, Dornan A T, Dow J A T and Davies S A, Renal neuroendocrine control of desiccation and cold tolerance by Drosophila suzukii. Pest Manag Sci 74: 800-810 (2017).
Predel R, Wegener C, Biology of the CAPA peptides in insects. Cell Mol Life Sci 63: 2477-2490 (2006).
Lamango N S, Nachman R J, Hayes T K, Strey A and Isaac R E, Hydrolysis of insect neuropeptides by an angiotensin converting enzyme from the housefly, M. domestica. Peptides 18: 47-52 (1997).
Terhzaz S, Cabrero P, Robben J H, Radford J C, Hudson B D, Milligan G, Dow J A and Davies S A, Mechanism and function of Drosophila capa GPCR: a desiccation stress-responsive receptor with functional homology to human neuromedinU receptor. PLoS One 7(1): e29897 (2012).
Blackman R L and Eastop V F, Aphids on the World's Crops: An Identification Guide, John Wiley & Sons Ltd, Chichester, U K. (2000).
Dow J A T, Maddrell S H P, Gortz A, Skaer N J V, Brogan S and Kaiser K, The Malpighian tubules of Drosophila melanogaster—a novel phenotype for studies of fluid secretion and its control. J Exp Biol 197: 421-428 (1994).
Davies S A, Huesmann G R, Maddrell S H, O'Donnell M J, Skaer N J, Dow J A T and Tublitz N J, CAP2b, a cardioacceleratory peptide, is present in Drosophila and stimulates tubule fluid secretion via cGMP. Am J Physiol 269: R₁₃₂₁-R1326 (1995).
Beyenbach K W, Skaer H and Dow J A, The developmental, molecular, and transport biology of Malpighian tubules. Annu Rev Entomol 55: 351-374 (2010).
Sadeghi A., Van Damme, E. J. M. and Smagghe G. (2009) Evaluation of the susceptibility of the pea aphid, Acyrthosiphon pisum, to a selection of novel biorational insecticides using an artificial diet. Journal of Insect Science 9:65.
Van Emden F (2009). Artificial diet for aphids—thirty years' experience. REDIA, XCII: 163-167

For standard molecular biology techniques, see Sambrook, J., Russel, D. W. Molecular Cloning, A Laboratory Manual. 3 ed. 2001, Cold Spring Harbor, New York: Cold Spring Harbor Laboratory Press

The features disclosed in the foregoing description, or in the following claims, or in the accompanying drawings, expressed in their specific forms or in terms of a means for performing the disclosed function, or a method or process for obtaining the disclosed results, as appropriate, may, separately, or in any combination of such features, be utilised for realising the invention in diverse forms thereof.

While the invention has been described in conjunction with the exemplary embodiments described above, many equivalent modifications and variations will be apparent to those skilled in the art when given this disclosure. Accordingly, the exemplary embodiments of the invention set forth above are considered to be illustrative and not limiting. Various changes to the described embodiments may be made without departing from the spirit and scope of the invention.

For the avoidance of any doubt, any theoretical explanations provided herein are provided for the purposes of improving the understanding of a reader. The inventors do not wish to be bound by any of these theoretical explanations.

Any section headings used herein are for organizational purposes only and are not to be construed as limiting the subject matter described.

Throughout this specification, including the claims which follow, unless the context requires otherwise, the word “comprise” and “include”, and variations such as “comprises”, “comprising”, and “including” will be understood to imply the inclusion of a stated integer or step or group of integers or steps but not the exclusion of any other integer or step or group of integers or steps.

It must be noted that, as used in the specification and the appended claims, the singular forms “a,” “an,” and “the” include plural referents unless the context clearly dictates otherwise. Ranges may be expressed herein as from “about” one particular value, and/or to “about” another particular value. When such a range is expressed, another embodiment includes from the one particular value and/or to the other particular value. Similarly, when values are expressed as approximations, by the use of the antecedent “about,” it will be understood that the particular value forms another embodiment. The term “about” in relation to a numerical value is optional and means for example+/−10%.

### Summary of Experimental Section

Hemipteran insect species including M. persicae contain three CAPA peptide sequences, CAPA-1, CAPA-2 and CAPA-3 (see Table 1).

CAPA-3, conserved in all hemipteran insects for which there is sequence information, has been shown to activate a Pyrokinin receptor in Brown Marmorated Stinkbug, H. halys (Ahn et al., 2020).

We have designed and synthesised an aphicidal CAPA-3 analogue which displays high efficacy against adult aphids; as well as nymphicidal activity.

### Methods

### General Procedures

All amino acids are of L-configuration unless otherwise stated. Standard Fmoc-protected amino acids were purchased from CEM Corporation or Pepceuticals. Suppliers of specialist amino acids are indicated as and when appropriate, as is peptide synthesis resin supplier. Peptide-grade DMF was purchased from Rathburn.

Peptides were synthesised on a Biotage Initiator+Alstra microwave-assisted peptide synthesiser or a CEM Liberty Prime microwave-assisted peptide synthesiser as specified.

High-resolution mass spectrometry (HRMS) was performed on a Bruker microTOF-Q II (ESI+).

Peptides were purified on a reverse-phase Dionex HPLC system equipped with Dionex P680 pumps and a Dionex UVD170U UV-vis detector (monitoring at 214 nm and 280 nm), using a Phenomenex, Gemini, C18, 5 μm, 250×21.2 mm column. Gradients were performed using solvents consisting of A (H₂O+0.1% TFA) and B (MeCN+0.1% TFA) and fractions were lyophilised on a Christ Alpha 2-4 LO plus freeze dryer.

Pure peptides were analysed on a Shimadzu reverse-phase HPLC (RP-HPLC) system equipped with Shimadzu LC-20AT pumps, a SIL-20A autosampler and a SPD-20A UV-vis detector (monitoring at 214 nm and 280 nm) using a Phenomenex, Aeris, 5 μm, peptide XB—C18, 150×4.6 mm column at a flow rate of 1 mL/min. RP-HPLC gradients were run using a solvent system consisting of solution A (100% H₂O+0.1% TFA) and B (100% MeCN+0.1% TFA). Typically, two gradients were used to characterise each peptide; a gradient from 5% to 95% solution B over 20 min (incorporating a 2 min hold at 5% solution B and a 5 min wash at 95% solution B at the start and end of the gradient respectively) and a gradient from 5-95% solution B over 50 min (incorporating a 5 min hold at 5% solution B and a 5 min wash at 95% solution B at the start and end of the gradient respectively). In some cases, specialised gradients were used and this is indicated where appropriate. Analytical RP-HPLC data is reported as column retention time (tR) in minutes (min). Analytical columns were maintained at ambient temperature.

LC-MS analysis was performed on a Thermo Scientific LCQ Fleet quadropole mass spectrometer of m/z range 50-2000 Da with an ESI source coupled to a Dionex Ultimate 3000 LC. Analyses were performed on a ReprosilGold 120 C18, 3 μm 150×4 mm column using a linear gradient of buffer A (95/5 H₂O/MeCN with 0.1% v/v TFA) to buffer B (95/5 MeCN/H₂O with 0.1% v/v TFA) over 20 min (incorporating a 2 min hold at 0% solution B and a 5 min wash at 100% solution B at the start and end of the gradient respectively). In cases where a specialised analytical RP-HPLC gradient was used to characterise a compound, an analogous LC-MS gradient was used. Analytical RP-HPLC and LC-MS samples were injected as 25 μL of a stock with concentration of 1 mg/mL in H₂O/MeCN with 0.1% v/v TFA. LC-MS column oven temperature was maintained at 30° C.

High-resolution mass spectrometry (HRMS) of pure peptides was performed on a Bruker microTOF-Q II (ESI+).

Proton nuclear magnetic resonance spectra (¹H NMR) for the purpose of peptide content calculations were recorded on an AVANCE III 400 Bruker (400 MHz). Proton chemical shifts are expressed in parts per million (ppm, 6 scale) and are referenced to residual protium in the NMR solvent (CDCl₃, δ 7.26; CD₃OD, δ 3.31 and D₂O, δ 4.79). The following abbreviations were used to describe peak patterns when appropriate: br=broad, s=singlet, d=doublet, t=triplet, q=quadruplet, m=multiplet. Coupling constants, J, are reported in Hertz unit (Hz).

### General Procedure for Automated Peptide Synthesis

### Biotage Initiator+Alstra Synthesiser:

Fmoc-protected amino acids were prepared as a 0.2 M (0.1 mmol syntheses), 0.5 M (0.2 mmol syntheses) or 0.7 M (0.5 mmol syntheses) solution in DMF. 5 equivalents of amino acid (relative to the resin loading) were used during coupling cycles. Oxyma and diisopropyl carbodiimide (DIC) were prepared as 0.2 M (0.1 mmol syntheses), 0.5 M (0.2 mmol syntheses) or 0.7 M (0.5 mmol syntheses) solutions in DMF. 5 equivalents of Oxyma and 5 equivalents of DIC (relative to resin loading) were used during coupling cycles. For Fmoc-deprotections, a solution of 20% morpholine (with 5% formic acid) in DMF was used. Coupling reactions were performed under microwave heating at 90° C. for 2 min with the exception of Fmoc-Cys(Trt)-OH, Fmoc-His(Trt)-OH and Fmoc-Arg(PBf)-OH. Coupling of Fmoc-Cys(Trt) and Fmoc-His(Trt)-OH was performed for 10 min at 50° C. Coupling of Fmoc-Arg(Pbf)-OH was performed for 2 successive cycles (double-coupled) for 2 min at 90° C. Microwave-assisted Fmoc deprotections were carried out at 90° C. for 1 min.

### CEM Liberty Blue Synthesiser

Fmoc-protected amino acids were prepared as a 0.2 M solution in NBP (Tamisolve). 5 equivalents of amino acid (relative to the resin loading) were used during coupling cycles. Oxyma was prepared as a 0.5 M solution in NBP. DIC was prepared as 5 M solution in NBP. 5 equivalents of Oxyma and 5 equivalents of DIC (relative to resin loading) were used during coupling cycles. For Fmoc-deprotections, a solution of 20% pyrrolidine was used. Coupling reactions and Fmoc-deprotections were performed under microwave heating at 90° C. for 2 min and 1 min respectively with the exception of Fmoc-Cys(Trt)-OH, Fmoc-His(Trt)-OH and Fmoc-Arg(Pbf)-OH. Coupling of Fmoc-Cys(Trt) and Fmoc-His(Trt)-OH was performed for 5 min at 50° C. Coupling of Fmoc-Arg(Pbf)-OH was performed for 2 successive cycles of 5 min at 75° C.

### General Procedure for TFA Cleavage of Peptides

Typically, cleavage tests of peptides were performed by taking ~3 mg of dried resin beads and treating them with TFA/TIS/water (95:2.5:2.5:) for 3 h. The filtrate was drained, concentrated and then triturated in cold diethyl ether (Et₂O). The triturate was dissolved in acetonitrile/water, and then analysed by RP-HPLC/LC-MS.

Peptides were typically cleaved from the resin in bulk by gently rocking the resin at rt in a cleavage cocktail of TFA/TIS/H₂O (95:2.5:2.5) for 3 h before being drained and the TFA blown off with a steady stream of N₂ gas. Peptides containing cysteine or tryptophan residues were cleaved from the resin using a cleavage cocktail of TFA/TIS/H₂O/DODT (94:2.5:1:2.5) for 3 h. In all cases the crude peptide was triturated with cold Et₂O. Et₂O was removed from the resulting crude peptide pellet under a steady stream of nitrogen. The crude peptide was then redissolved in H₂O/MeCN and purified by RP-HPLC.

Synthesis of SB-P-15
SEQ ID 39
(SDSKNT[n-me-A]LWFGPRL-[NH₂])

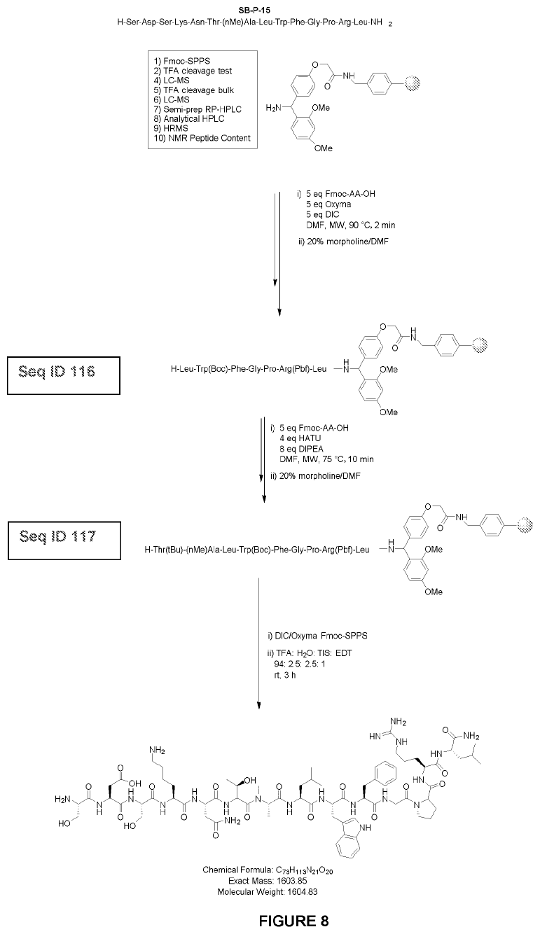

The synthesis of SB-P-15 was performed by Fmoc-SPPS on a Biotage Initiator+Alstra automated peptide synthesiser using microwave-assisted deprotection and coupling reactions (Scheme 1—FIG. 8). H-Rink Amide ChemMatrix resin (Biotage) was chosen in order to leave the desired C-terminal amide functionality upon TFA-mediated cleavage. A 20% solution of morpholine in DMF with 5% formic acid included as an additive was used for Fmoc-deprotection reactions. A diisopropylcarbodiimide (DIC)/ ethyl cyano(hydroxyimino) acetate (OxymaPure®) coupling strategy was used to reduce the risk of epimerisation during amide-coupling steps throughout with the exception of Fmoc-(NMe)Ala-OH and Fmoc-Thr(Boc)-OH. For these residues, an HATU/DIPEA coupling strategy was used in an effort to overcome the steric-hindrance inherent to coupling of N-methylated building blocks and the residue immediately following. Simultaneous cleavage from the resin and side-chain deprotection was achieved using a cleavage cocktail of trifluoroacetic acid (TFA): triisopropyl silane (TIS): H₂O: ethane dithiol (EDT) (94:2.5:2.5:1) for 3 h.

Following TFA-mediated resin-cleavage and side-chain deprotection and RP-HPLC purification of the crude peptide, SB-P-15 was obtained in 90% purity and 3% overall yield.

Synthesis of SB-P-28
SEQ ID 152
(SDSKNT[Peptoid-Phe]LWFGPRL-[NH₂])

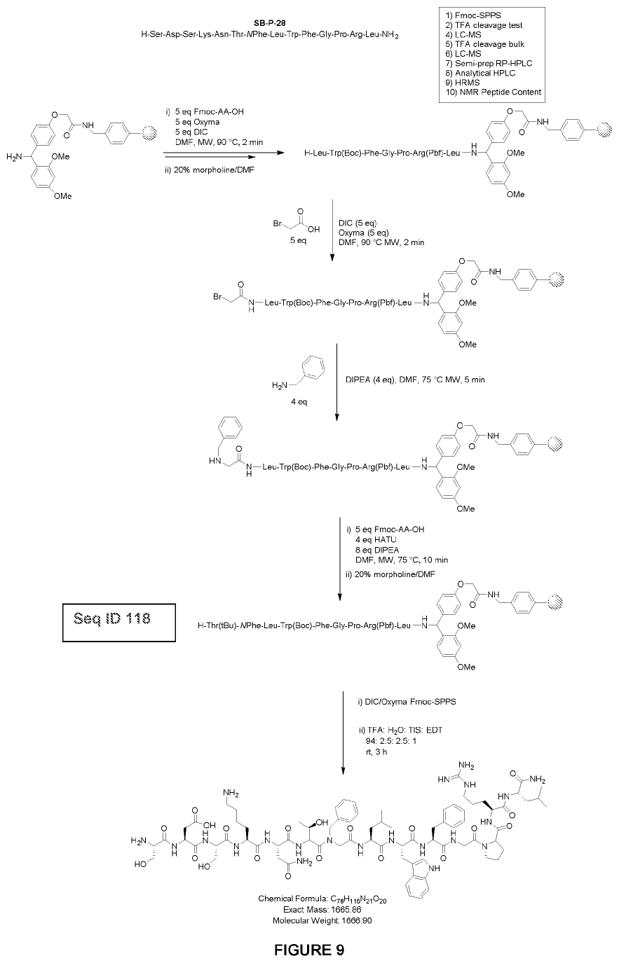

SB-P-28 is a Capa-3 analogue with an Ala7N-Phe mutation. Installation of peptoid residues such as the proposed N-Phe is non-trivial, with no Fmoc-peptoid building blocks commercially available. With this in mind, a synthetic route was designed in which this moiety is installed via a two-step process in automation (Scheme 2—FIG. 9).

LC-MS analysis indicated that synthesis of the SB-P-28 using this strategy had proceeded well. Following RP-HPLC purification, the desired peptide was obtained in 93% purity and 5% overall yield.

Synthesis of SB-P-29
SEQ ID 52
([ind]-SDSKNT[n-me-A]LWFGPRL-[NH₂])

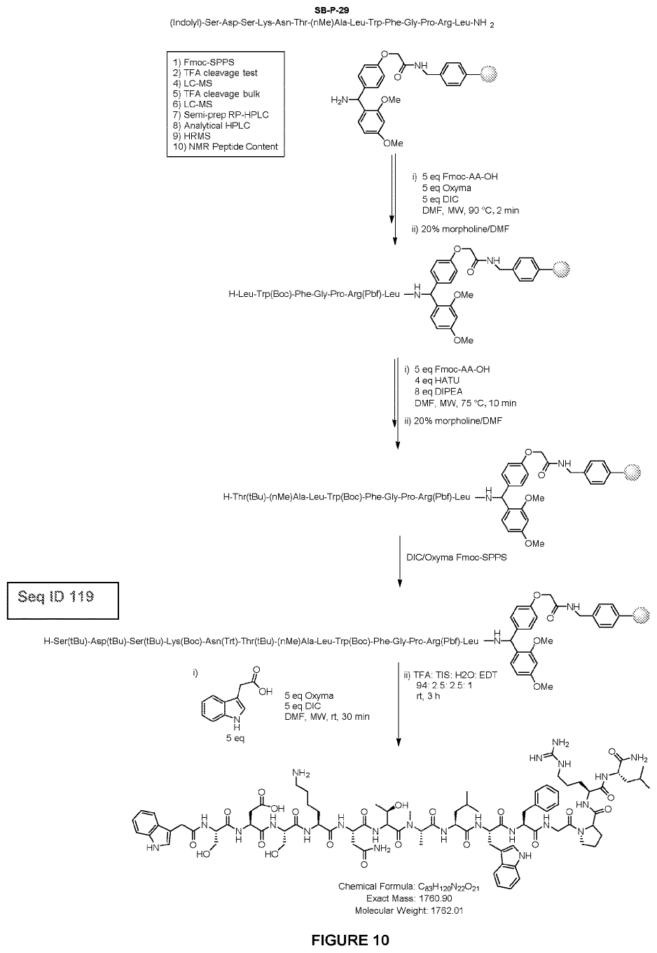

The synthesis of this peptide was performed on Tentagel S RAM resin (f=0.22 mmol/g) by automated Fmoc-SPPS using the Biotage Initiator Alstra peptide synthesiser (Scheme 3—FIG. 10).

Coupling steps were carried out using 4 eq (relative to resin loading) of Fmoc-amino acid (0.2 M) and DIC/Oxyma (0.5 M) for 2 min at 90° C. Fmoc-deprotection was effected in 20% morpholine/DMF (with 5% formic acid additive) for 1 min at 90° C. Double-coupling steps were employed for Leu8, Trp9, Arg13 and Leu14. Coupling using 4 eq HATU and 8 eq DIPEA for 10 min at 75° C. was used for (NMe)Ala7 and Thr6 to overcome the steric-hindrance associated with coupling of and to N-methyl residues. Installation of the N-terminal indole moiety was achieved by coupling of 3-indoleacetic acid (5 eq) with DIC/Oxyma (5 eq) for 30 min at room temperature.

Synthesis of SB-P-30
SEQ ID 53
([glucosamine-Suc]-SDSKNT[n-me-A]LWFGPRL-[NH₂]

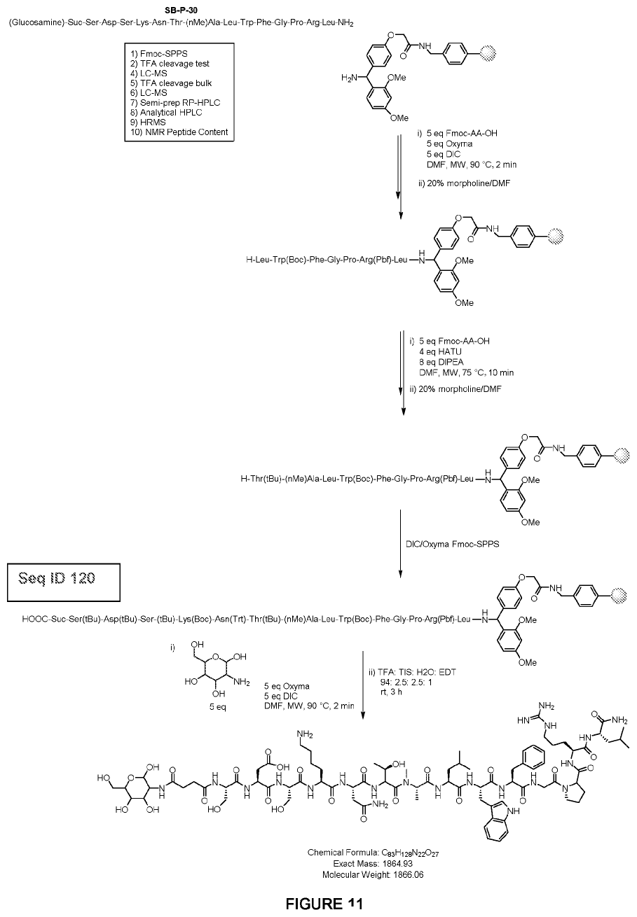

The synthesis of this peptide was performed on Tentagel S RAM resin (f=0.22 mmol/g) by automated Fmoc-SPPS using the Biotage Initiator Alstra peptide synthesiser (Scheme 4—FIG. 11).

Coupling steps were carried out using 4 eq (relative to resin loading) of Fmoc-amino acid (0.2 M) and DIC/Oxyma (0.5 M) for 2 min at 90° C. Fmoc-deprotection was effected in 20% morpholine/DMF (with 5% formic acid additive) for 1 min at 90° C. Double-coupling steps were employed for Leu8, Trp9, Arg13 and Leu14. Coupling using 4 eq HATU and 8 eq DIPEA for 10 min at 75° C. was used for (NMe)Ala7 and Thr6 to overcome the steric-hindrance associated with coupling of and to N-methyl residues. Installation of the N-terminal glucosamine moiety was achieved first by coupling of succinic acid (5 eq) with DIC/Oxyma (5 eq) for 2 min at 90° C. as a spacing residue, followed by coupling of glucosamine (5 eq) with DIC/Oxyma (5 eq) for 2 min at 90° C.

Synthesis of SB-P-31
SEQ ID 54
((biotin)-SDSKNT[n-me-A]LWFGPRL-[nh₂])

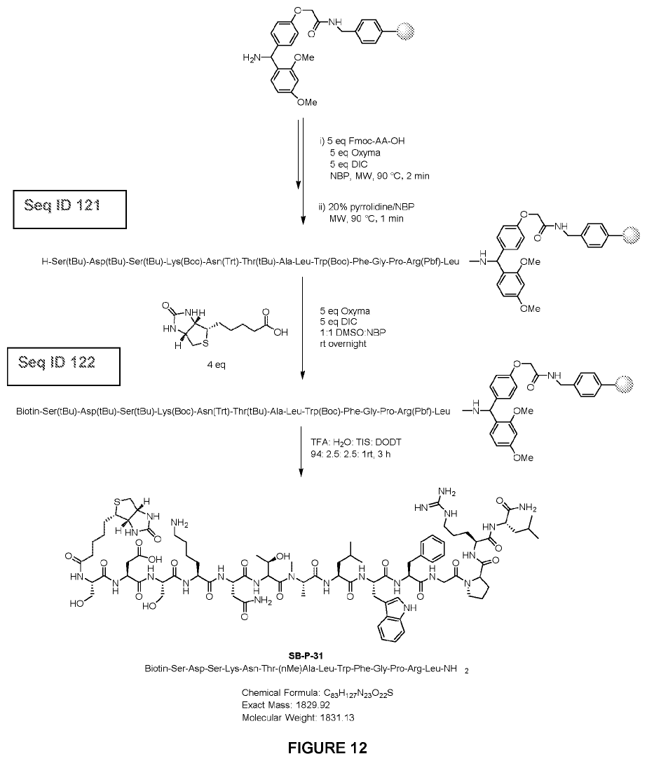

The synthesis of this peptide was performed on Rink Amide ProTide resin (f=0.19 mmol/g) by automated Fmoc-SPPS using the CEM Liberty Blue peptide synthesiser (Scheme 5—FIG. 12).

Coupling steps were carried out using 5 eq (relative to resin loading) of Fmoc-amino acid (0.2 M) and DIC/Oxyma (0.5 M) for 2 min at 90° C. Fmoc-deprotection was effected in 20% pyrrolidine (with 0.1 M Oxyma additive) for 1 min at 90° C. Double-coupling steps were employed for Leu8, Trp9, Arg13 and Leu14. Coupling using 5 eq DIC/Oxyma for 4 min at 90° C. was used for (NMe)Ala7 and Thr6 to overcome the steric-hindrance associated with coupling of and to N-methyl residues. Installation of the N-terminal biotin moiety was achieved by coupling of D-Biotin (5 eq) with DIC/Oxyma (5 eq) overnight at room temperature.

### Bioinformatics, Peptide Sequences for Selectivity

Hemipteran insect species including M. persicae contain 3 CAPA peptide sequences, below, Table 1. Interrogation of the DiNER database of insect neuropeptides (Yeoh et al., Insect Biochem and Mol Biol, 2018), Table 1 below, indicates CAPA-3 sequence. This is distinct from CAPA-1 and CAPA-2 within the Hemiptera and other insect orders, e.g. Diptera, not just due to the C-terminus PRL motif but also the GPRL motif. This motif is conserved across Hemiptera (Table 2).

TABLE 1
Sequences of M. persicae CAPA peptide sequences.

| | | Species | Amino Acid Sequence | | |
|---|---|---|---|---|---|
| Isoform | Neuropeptide | Name | [symbol] | | |
| Myzpe-CAPA- | CAPA [symbol] | Myzus persicae | ESVAGLIPFPRVamide | | SEQ ID 78 |
| 1 | | | | | |
| Myzpe-CAPA- | CAPA [symbol] | Myzus persicae | EGLIPFPRIamide | | SEQ ID 79 |
| 2 | | | | | |
| Myzpe-CAPA- | CAPA [symbol] | Myzus persicae | SDSKNTALWFGPRLamide | | SEQ ID 80 |
| 3 | | | | | |
| | | Species | Amino Acid Sequence | Molecular | |
| Isoform | Neuropeptide | Name | [symbol] | Weight [symbol] | |
| [symbol] | CAPA [symbol] | [symbol] | [symbol] | [symbol] | SEQ ID 81 |
| [symbol] | | [symbol] | | | |
| [symbol] | CAPA [symbol] | [symbol] | [symbol] | [symbol] | SEQ ID 82 |
| [symbol] | | [symbol] | | | |
| [symbol] | CAPA [symbol] | [symbol] | [symbol] | [symbol] | SEQ ID 83 |
| [symbol] | | [symbol] | | | |
| [symbol] | CAPA [symbol] | [symbol] | [symbol] | [symbol] | SEQ ID 84 |
| [symbol] | | | | | |
| [symbol] | CAPA [symbol] | [symbol] | [symbol] | [symbol] | SEQ ID 85 |
| [symbol] | | | | | |
| [symbol] | CAPA [symbol] | [symbol] | [symbol] | [symbol] | SEQ ID 86 |
| [symbol] | | | | | |
| [symbol] | CAPA [symbol] | [symbol] | [symbol] | [symbol] | SEQ ID 87 |
| [symbol] | | | | | |
| [symbol] | CAPA [symbol] | [symbol] | [symbol] | [symbol] | SEQ ID 88 |
| [symbol] | | | | | |
| [symbol] | CAPA [symbol] | [symbol] | [symbol] | [symbol] | SEQ ID 89 |
| [symbol] | | | | | |
| [symbol] | CAPA [symbol] | [symbol] | [symbol] | [symbol] | SEQ ID 90 |
| [symbol] | | | | | |
| [symbol] | CAPA [symbol] | [symbol] | [symbol] | [symbol] | SEQ ID 91 |
| [symbol] | | | | | |
| [symbol] | CAPA [symbol] | [symbol] | [symbol] | [symbol] | SEQ ID 92 |
| [symbol] | | | | | |
| [symbol] | CAPA [symbol] | [symbol] | [symbol] | [symbol] | SEQ ID 93 |
| [symbol] | | | | | |
| [symbol] | CAPA [symbol] | [symbol] | [symbol] | [symbol] | SEQ ID 94 |
| [symbol] | | [symbol] | | | |
| [symbol] | CAPA [symbol] | [symbol] | [symbol] | [symbol] | SEQ ID 95 |
| [symbol] | | [symbol] | | | |
| [symbol] | CAPA [symbol] | [symbol] | [symbol] | [symbol] | SEQ ID 96 |
| [symbol] | | [symbol] | | | |
| [symbol] | CAPA [symbol] | [symbol] | [symbol] | [symbol] | SEQ ID 97 |
| [symbol] | | [symbol] | | | |
| [symbol] | CAPA [symbol] | [symbol] | [symbol] | [symbol] | SEQ ID 98 |
| [symbol] | | | | | |
| [symbol] | CAPA [symbol] | [symbol] | [symbol] | [symbol] | SEQ ID 99 |
| [symbol] | | [symbol] | | | |
| [symbol] | CAPA [symbol] | [symbol] | [symbol] | [symbol] | SEQ ID 100 |
| [symbol] | | [symbol] | | | |
| [symbol] | CAPA [symbol] | [symbol] | [symbol] | [symbol] | SEQ ID 101 |
| [symbol] | | | | | |
| [symbol] | CAPA [symbol] | [symbol] | [symbol] | [symbol] | SEQ ID 102 |
| [symbol] | | | | | |
| [symbol] | CAPA [symbol] | [symbol] | [symbol] | [symbol] | SEQ ID 103 |
| [symbol] | | [symbol] | | | |
| [symbol] | CAPA [symbol] | [symbol] | [symbol] | [symbol] | SEQ ID 104 |
| [symbol] | | | | | |
| [symbol] | CAPA [symbol] | [symbol] | [symbol] | [symbol] | SEQ ID 105 |
| [symbol] | | [symbol] | | | |
| [symbol] | CAPA [symbol] | [symbol] | [symbol] | [symbol] | SEQ ID 106 |
| [symbol] | | [symbol] | | | |
| [symbol] | CAPA [symbol] | [symbol] | [symbol] | [symbol] | SEQ ID 107 |
| [symbol] | | [symbol] | | | |
| [symbol] | CAPA [symbol] | [symbol] | [symbol] | [symbol] | SEQ ID 108 |
| [symbol] | | [symbol] | | | |
| [symbol] | CAPA [symbol] | [symbol] | [symbol] | [symbol] | SEQ ID 109 |
| [symbol] | | | | | |
| [symbol] | CAPA [symbol] | [symbol] | [symbol] | [symbol] | SEQ ID 110 |
| [symbol] | | [symbol] | | | |
| [symbol] | CAPA [symbol] | [symbol] | [symbol] | [symbol] | SEQ ID 111 |
| [symbol] | | [symbol] | | | |
| [symbol] | CAPA [symbol] | [symbol] | [symbol] | [symbol] | SEQ ID 112 |
| [symbol] | | [symbol] | | | |
| [symbol] | CAPA [symbol] | [symbol] | [symbol] | [symbol] | SEQ ID 113 |
| [symbol] | | [symbol] | | | |

[symbol]

indicates data missing or illegible when filed

TABLE 2
CAPA-3 sequences across Hemipteran species (DiNer database)
Accordingly, the CAPA-3 peptide was investigated as a new potential biocide against
hemipteran species including aphids.
Results
Endogenous CAPA-3 (SB-P-1) and 2 biostable analogues (SB-P-14; SB-P-15)

| Code | Sequence | Species | Family | Mwt | |
|---|---|---|---|---|---|
| SB-P-1 | Hy-SDSKNTALWFGPRL-[NH₂] | Aphids, | CAPA- | 1590.55 | SEQ ID 114 |
| | | stinkbugs, | 3 | | |
| | | hemiptera | | | |
| SB-P-14 | Hy-Ahx-SDSKNT[n-me- | Aphids, | CAPA- | 1974.3 | SEQ ID 39 |
| | A]LWFGPRL-[NH₂] | stinkbugs, | 3 | | |
| | | hemiptera | | | |
| SB-P-15 | Hy-SDSKNT[n-me- | Aphids, | CAPA- | 1604.52 | SEQ ID 40 |
| | AILWFGPRL-[NH₂] | stinkbugs, | 3 | | |
| | | hemiptera | | | |

### Table 3. Sequences and Properties of Endogenous Hemipteran CAPA-3 and Two Peptide Analogues Thereof.

Peptide efficacy (2×10⁻⁴ M) was assessed on M. persicae (peach potato/Green Peach Aphid) using IRAC leaf dip assays on adult aphids. Peptides were administered on leaves either in DDH₂O or Amino Acid Mix (Sadeghi et al., 2010) to increase uptake via feeding. Insecticide treatment was with Spirotetramat, 2×10⁻⁴ M

Lethality of adult aphids was scored 72, 96 and 120 hours; number of nymphs was counted at 72, 96 and 120 hours, where maximal efficacy was observed at 120 hours.

Although SB-P-14 did not perform as well as endogenous CAPA-3, SB-P-15 was found to be a highly effective aphicidal and nymphicidal peptide; and an improvement on endogenous CAPA-3 efficacy.

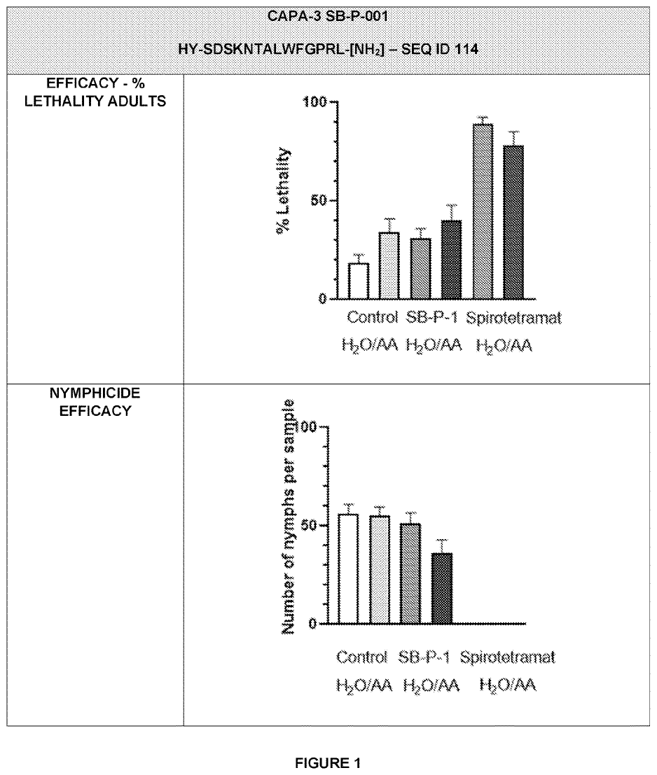

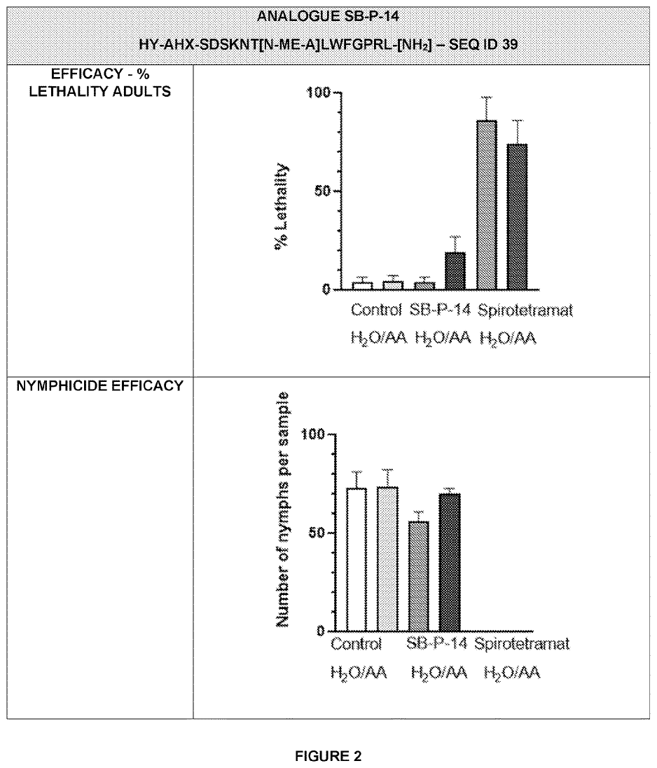

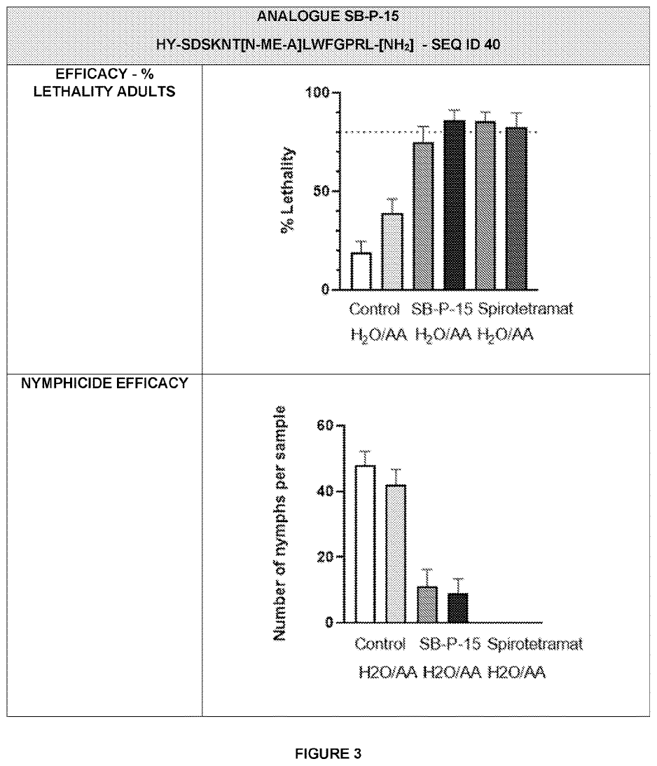

Results are shown in FIGS. 1 to 3.

Data are from 120 hours post-treatment, indicating % adult lethality (left panel); and number of nymphs per sample (right panel) for each of SB-P-1 (endogenous CAPA-3, N =26 (H₂O); 15 (AA)), SB-P-14 (CAPA-3 analogue, N=5); SB-P-15 (CAPA-3 analogue, N=10). Peptide application rates are 2×10⁻⁴ M. Note that nymph values for spirotetramat are 0.

### Co-Application

SB-P-15 has been Tested in Co-Application with 2315, SB-P-9 and AH59

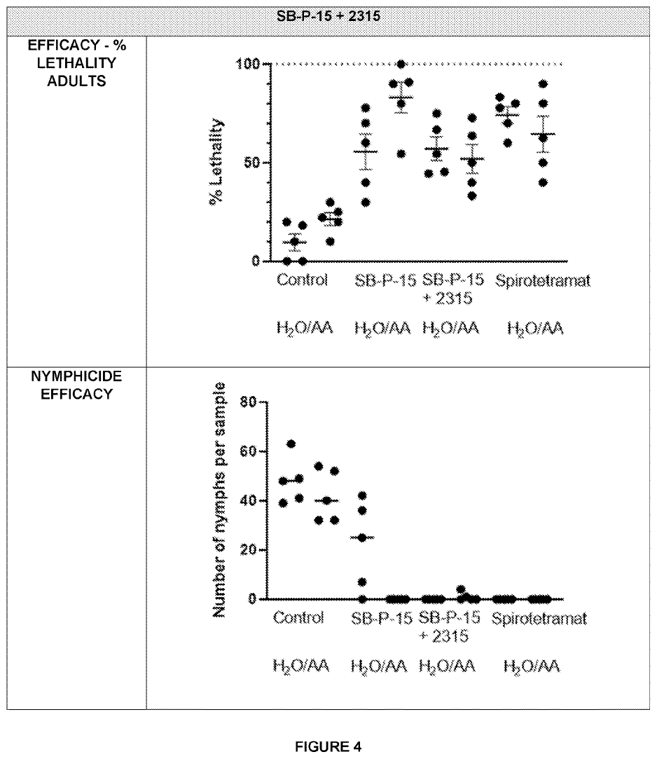

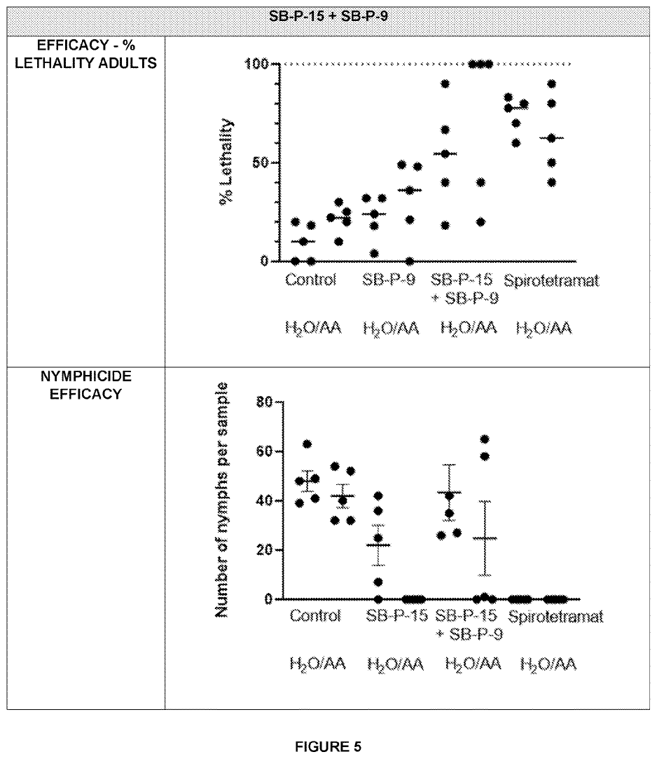

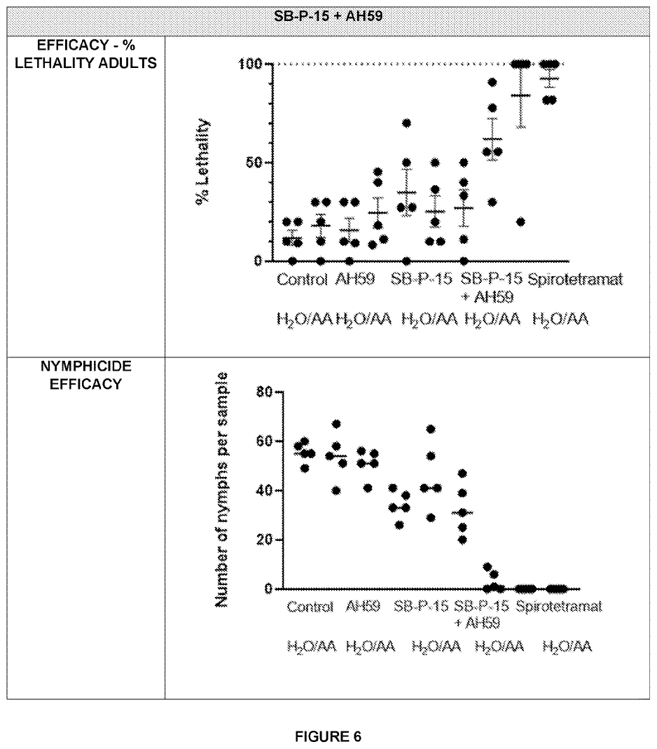

Results are shown in FIGS. 4 to 6. Data are for 120 hours, indicating % adult lethality (left panel); and number of nymphs per sample (right panel). Individual data points are shown, N=5. Peptide application rates are 2×10⁻⁴ M.

Co-application of SB-P-15/2315 results in 100% nymphicidal efficacy.

Co-application of SB-P-15/SB-P-9 shows high efficacy against adult aphids and also nymphicidal efficacy in the presence of amino acids.

Co-application of SB-P-15/AH59 shows high efficacy against adult aphids and near 100% nymphicidal efficacy in the presence of amino acids.

### CAPA-3 Mode of Action

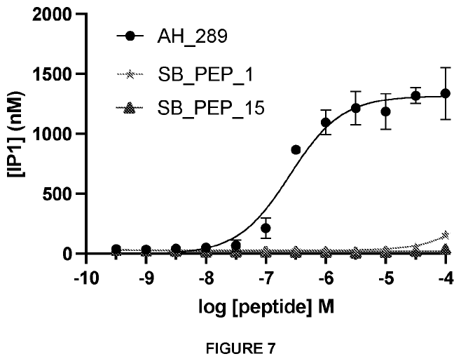

Initial screen against M. persicae CAPA receptor indicates that CAPA-3 and SB-P-15 do not activate this receptor, FIG. 7, in agreement from data in Ahn et al, 2020 where CAPA-3 does not activate H. halys CAPA receptor but instead a pyrokinin receptor isoform.

### Efficacy Data

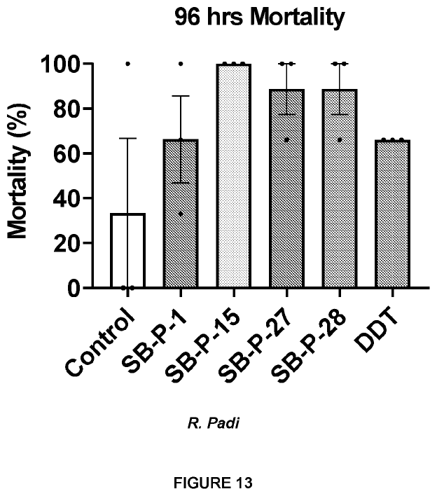

Efficacy data of further peptides is provided in FIGS. 13, 14 and 15.

FIG. 13 shows efficacies of different Capa-3 analogues against R. padi in a leaf dip assay. The following peptides were tested:

SB-P-001:
SEQ ID 114
Hy-SDSKNTALWFGPRL-[nh₂]
SB-P-015:
SEQ ID 40
Hy-SDSKNT[n-me-A]LWFGPRL-[nh₂]
SB-P-027:
SEQ ID 48
Hy-[Peg8]-SDSKNT[n-me-A]LWFGPRL-[nh₂]
SB-P-028:
SEQ ID 49
Hy-SDSKNT[Peptoid-Phe]LWFGPRL-[nh₂]

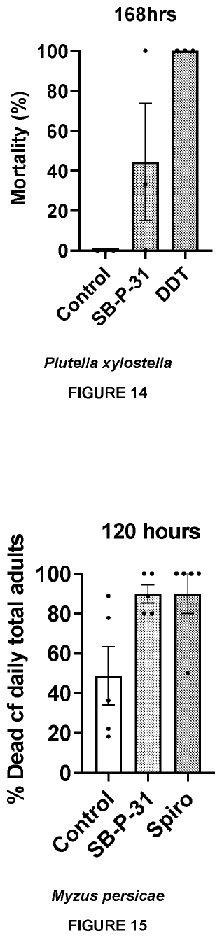

FIG. 14 shows efficacies of different Capa-3 analogues against R. padi in a leaf dip assay. The following peptide was tested:

SB-P-31:
SEQ ID 54
Hy-(biotin)-SDSKNT[n-me-A]LWFGPRL-[nh₂]

FIG. 15 shows efficacies of different Capa-3 analogues against M. persicae in a leaf dip assay. The following peptide was tested:

SB-P-31:
SEQ ID 54
Hy-(biotin)-SDSKNT[n-me-A]LWFGPRL-[nh₂]

### NUMBERED PARAGRAPHS

The following paragraphs define particular aspects and embodiments of the invention.
1. An insecticidal compound having the formula (I) below, or a salt or solvate thereof:

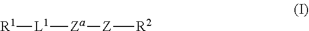

wherein:
R¹ is hydrogen (which may be designated “H—” or “Hy-”), C₁₋₄alkyl (e.g. methyl, ethyl, propyl, butyl), formyl, —N(R^{1a})—C(═N⁺(R^{1b})(R^{1c}))NR^{1d}R^{1e}, —C(═N⁺—(R^{1b})(R^{1c}))NR^{1d}R^{1e}, acyl, fatty acyl, benzyl, benzoyl, heteroaryl or trifluoroacetyl, —NHC₁₋₁₈alkyl, —NHC₆₋₁₆Aryl, —NH—C₁₋₆alkyl-C₆₋₁₀aryl, biotin, a sugar moiety or (poly)alkyleneglycol;
wherein each of R^{1a}, R^{1b}, R^{1c}, R^{1d} and R^{1e} is independently selected from hydrogen or C₁₋₄ alkyl (e.g. methyl, ethyl, propyl, butyl);
and any alkyl, formyl, acyl, fatty acyl, benzyl, benzoyl, heteroaryl or trifluoroacetyl, —NHC₁₋₁₈alkyl, —NHC₆₋₁₆Aryl, —NH—C₁₋₆alkyl-C₆₋₁₀aryl or (poly)alkyleneglycol may optionally be substituted with one or more groups selected from halogen, C₁₋₆alkyl, C₁₋₆haloalkyl or a sugar moiety;
L¹ is absent or is:
   —(C═O)C₁₋₁₆-alkylene-NH— where * denotes the point of attachment to Z;
   (C₁₋₂₀)alkylene,
   (C₂₋₂₀)alkenylene,
   (poly)alkyleneglycol;
   wherein L¹ is optionally substituted with one or more groups selected from oxo (═O), halogen, ═N and ═S;
Z^{a} is peptide comprising from 1 to 12 amino acids; wherein Z^{a} optionally comprises a (poly)alkyleneglycol linker in the peptide sequence;
Z is a peptide of the formula:

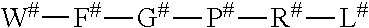

wherein “^{#}” indicates that the residues “W^{#}”, “F^{#}”, “G^{#}”, “P^{#}”, “R^{#}” and “L^{#}” are independently an unmodified amino acid, a modified amino acid or non-natural amino acid analogue; and
at least one of the residues in peptides Z^{a} and Z is a modified amino acid or non-natural amino acid analogue; and
R² is NH₂, NR^{2a}H, NR^{2a}R^{2b}, or OR^{2a}; wherein each of R^{2a} and R^{b} if present are independently C₁₋₆-alkyl (e.g. methyl, ethyl, propyl, butyl, pentyl or hexyl), C₃₋₆-alkenyl, C₆₋₁₆-aryl, C₆₋₁₆-aryl-C₁₋₆-alkyl, C₁₋₆-alkylene-C₆₋₁₆-aryl, or C₁₋₆haloalkyl, each of which may optionally be substituted with one or more groups selected from halogen, C₁₋₆-alkyl, or C₁₋₆haloalkyl.
2. The insecticidal compound according to paragraph 1, wherein if R¹-L¹- is:

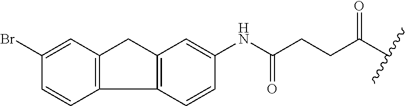 [SMILES: *C(=O)CCC(=O)Nc1ccc2c(c1)Cc1cc(Br)ccc1-2]

then at least one modified amino acid residue or non-natural amino acid residue in peptide Z^{a} or Z is not:
a beta amino acid; or
a D-configuration amino acid.
3. The insecticidal compound according to paragraph 1, wherein at least one modified amino acid residue or non-natural amino acid residue in peptide Z^{a} or Z is not:
   a beta amino acid,
   a D-configuration amino acid,
   Hyp, Oic, Sar, Nle, or Aib.
4. The insecticidal compound according to any of paragraphs 1 to 3, wherein at least one of the residues in peptides Z^{a} and Z is:
   i. an N-methylated amino acid;
   ii. a peptoid amino acid analogue;
   iii. a sugar modified amino acid; or
   iv. a biotin modified amino acid.
5. The insecticidal compound according to any one of the preceding paragraphs, wherein at least one of the residues in peptides Z^{a} and Z is:
   i. an N-methylated amino acid;
   ii. a peptoid amino acid analogue.
6. The insecticidal compound according to any one of the preceding paragraphs, wherein at least one of the residues in peptides Z^{a} and Z is an N-methylated amino acid.
7. The insecticidal compound according to any one of the preceding paragraphs, wherein R¹ is selected from:
   hydrogen;
   acyl optionally substituted with a sugar moiety;
   fatty acyl;
   heteroaryl;
   NHC₆₋₁₆Aryl;
   a sugar moiety;
   biotin; or
   (poly)ethyleneglycol of the formula *—(OCH₂CH₂)ₙ—Rₚ, where * denotes the point of attachment to L¹ or Z, n is an integer from 1 to 16, and Rₚ is selected from —NH₂, —OH or —OMe.
8. The insecticidal compound according to any one of the preceding paragraphs, wherein R¹ is selected from:
   hydrogen;
   acyl optionally substituted with a sugar moiety, wherein the acyl group is selected from formyl, acetyl (Ac), propanoyl, butanoyl;
   fatty acyl selected from palmitoyl, butyryl, cerotoyl, decanoyl, docosenoyl, dodecanoyl, eleostearoyl, heptanoyl, hexanoyl, icosanoyl, icosenoyl, lignoceroyl, linoleoyl, lipoyl, myristoleoyl, nonanoyl, octadecanoyl, ocatanoyl, palmitoleoyl, stearoyl, undecanoyl, and valeryl;
   indolyl;
   —NHC₆₋₁₆Aryl;
   a monosaccharide or disaccharide moiety;
   biotin;
   (poly)ethyleneglycol of the formula *—(OCH₂CH₂)ₙ—Rₚ, where * denotes the point of attachment to L¹ or Z, n is an integer from 4 to 12, and Rₚ is selected from —NH₂, —OH or —OMe.
9. The insecticidal compound according to any one of the preceding paragraphs, wherein R¹ is selected from:
   hydrogen;
   acetyl substituted with a monosaccharide or disaccharide moiety;
   palmitoyl;
   indolyl;
   —N(H)-fluorenyl substituted with one or more bromine atoms, e.g.

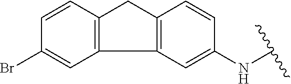 [SMILES: *Nc1ccc2c(c1)-c1cc(Br)ccc1C2]

(poly)ethyleneglycol of the formula *—(OCH₂CH₂)ₙ—OH, where * denotes the point of attachment to Z, and n is an integer from 6 to 10.
10. The insecticidal compound according to any one of the preceding paragraphs, wherein L¹ is absent or is:
   —(C═O)C₄₋₁₂-alkylene-NH—, where * denotes the point of attachment to Z;
   —(C═O)—C₁₋₄alkylene-(C═O)—.
11. The insecticidal compound according to any one of the preceding paragraphs, wherein L¹ is absent or is:

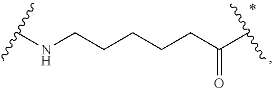 [SMILES: *NCCCCCC(*)=O]

where * denotes the point of attachment to Z; or

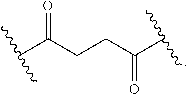 [SMILES: *C(=O)CCC(*)=O]

12. The insecticidal compound according to any one of the preceding paragraphs, wherein Z^{a} is a peptide of the formula:

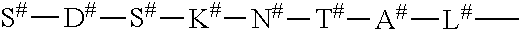

wherein “^{#}” indicates that the residue is either a naturally occurring amino acid, a modified amino acid or a non-natural amino acid analogue.
13. The insecticidal compound according to any one of the preceding paragraphs, wherein Z^{a}—Z is a peptide of the formula:

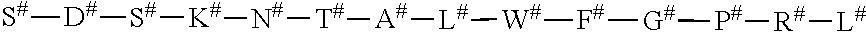

wherein one or more of the residues in Z^{a} or in Z is N-methylated.
14. The insecticidal compound according to any one of the preceding paragraphs, wherein the Z^{a} moiety has the formula:

| | |
|---|---|
| SDSKNTAL | SEQ ID 1 |
| SDSKNT[n-me-A]L | SEQ ID 2 |
| SDSKNT[Peptoid Phe]L | SEQ ID 3 |
| SDSKNT[Peptoid Ala]L | SEQ ID 4 |
| SDSKNTA[Peptoid Leu] | SEQ ID 5 |
| SDSKNTA[n-me-L] | SEQ ID 6 |
| [glucosamine-S]DSKNTAL | SEQ ID 7 |
| SD[glucosamine-S]KNTAL | SEQ ID 8 |
| SDSK[glucosamine-N]TAL | SEQ ID 9 |
| [glucosamine-Suc]-SDSKNTAL | SEQ ID 10 |
| [glucosamine-Suc]-SDSKNT[n-me-A]L | SEQ ID 11 |
| [Biotin]-SDSKNTAL | SEQ ID 12 |
| SDS[Biotin-K]NTAL | SEQ ID 13 |
| S[Biotin-D]SKNTAL | SEQ ID 153 |
| [Biotin]-SDSKNT[n-me-A]L | SEQ ID 15 |
| SDS[Biotin-K]NT[n-me-A]L | SEQ ID 16 |
| S[Biotin-D]SKNT[n-me-A]L | SEQ ID 154 |
| SDSKNT[Aib]L | SEQ ID 18 |
| SDSKNT[a]L; | SEQ ID 19 |
| SDSKNTA[l]; | SEQ ID 20 |
| SDSKNT[a][l] | SEQ ID 21 |
| SDSKNT[a]L | SEQ ID 22 |
| SDSKNTA[l] | SEQ ID 23 |
| SDSKNT[a][l]; | SEQ ID 24 |
| SDSKNT[B3A]L; | SEQ ID 25 |
| SDSKNTA[B3L]; or | SEQ ID 26 |
| SDSKNT[Aib]L. | SEQ ID 27 |

15. The insecticidal compound according to any one of the preceding paragraphs, wherein the Z moiety has the formula:

| | |
|---|---|
| WFGPRL; | SEQ ID 29 |
| W[Peptoid Phe]GPRL; | SEQ ID 30 |
| W[n-me-F]GPRL; | SEQ ID 31 |
| WFGPR[n-me-L]; | SEQ ID 32 |
| W[f]GPRL; | SEQ ID 33 |
| [w]FGPRL; | SEQ ID 34 |
| [w][f]GPRL; | SEQ ID 35 |
| W[B3F]GPRL; or | SEQ ID 36 |
| WFG[Hyp]RL. | SEQ ID 37 |

16. The insecticidal compound according to any one of the preceding paragraphs, wherein R² is NH₂.
17. A compound according to paragraph 1, having the formula:

| | |
|---|---|
| Hy-Ahx-SDSKNT[n-me-A]LWFGPRL-[NH₂] | SEQ ID 39 |
| Hy-SDSKNT[n-me-A]LWFGPRL-[NH₂] | SEQ ID 40 |
| Hy-SDSKNT[Peptoid-Phe]LWFGPRL-[NH₂] | SEQ ID 152 |
| Hy-SDSKNT[Peptoid-Ala]LWFGPRL-[NH₂] | SEQ ID 41 |
| Hy-SDSKNTA[Peptoid Leu]WFGPRL-[NH₂] | SEQ ID 42 |
| Hy-SDSKNTALW[Peptoid-Phe]GPRL-[NH₂] | SEQ ID 43 |
| Hy-SDSKNTA[n-me-L]WFGPRL-[NH₂] | SEQ ID 44 |
| Hy-SDSKNTALW[n-me-F]GPRL-[NH₂] | SEQ ID 45 |
| Hy-SDSKNTALWFGPR[n-me-L]-[NH₂] | SEQ ID 46 |
| Hy-SDSKNT[n-me-A]LWFGPR[n-me-L]-[NH₂] | SEQ ID 47 |
| Hy-[Peg8]-SDSKNT[n-me-A]LWFGPRL-[NH₂]; | SEQ ID 48 |
| Hy-[Palm]-SDSKNT[n-me-A]LWFGPRL-[NH₂]; | SEQ ID 49 |
| Hy-[ado]-SDSKNT[n-me-A]LWFGPRL-[NH₂]; | SEQ ID 50 |
| Hy-[2Abf-Suc]-SDSKNT[n-me-A]LWFGPRL-[NH₂]; | SEQ ID 51 |
| [Ind]-SDSKNT[n-me-A]LWFGPRL-[NH₂]; | SEQ ID 52 |
| Hy-[glucosamine-Suc]-SDSKNT[n-me-A]LWFGPRL-[NH₂] | SEQ ID 53 |
| Hy-[biotin]-SDSKNT[n-me-A]LWFGPRL-[NH₂] | SEQ ID 54 |
| Hy-[Biotin]-SDSKNTALWFGPRL-[NH₂] | SEQ ID 55 |
| Hy-SDS[Biotin-K]NTALWFGPRL-[NH₂] | SEQ ID 56 |
| Hy-S[Biotin-D]SKNTALWFGPRL-[NH₂] | SEQ ID 155 |
| Hy-SDS[Biotin-K]NT[n-me-A]LWFGPRL-[NH₂] | SEQ ID 58 |
| Hy-S[Biotin-D]SKNT[n-me-A]LWFGPRL-[NH₂] | SEQ ID 156 |
| Hy-SDSKNT[a]LWFGPRL-[NH₂]; | SEQ ID 60 |
| Hy-SDSKNTA[l]WFGPRL-[NH₂]; | SEQ ID 61 |
| Hy-SDSKNTALW[f]GPRL-[NH₂]; | SEQ ID 62 |
| Hy-SDSKNTAL[w]FGPRL-[NH₂]; | SEQ ID 63 |
| Hy-SDSKNT[a][l]WFGPRL-[NH₂]; | SEQ ID 64 |
| Hy-SDSKNT[a]LW[f]GPRL-[NH₂]; | SEQ ID 65 |
| Hy-SDSKNTA[l]W[f]GPRL-[NH₂]; | SEQ ID 66 |
| Hy-SDSKNT[a][l]W[f]GPRL-[NH₂]; | SEQ ID 67 |
| Hy-SDSKNT[a][l][w][f]GPRL-[NH₂]; | SEQ ID 68 |
| Hy-SDSKNT[B3A]LWFGPRL-[NH₂]; | SEQ ID 69 |
| Hy-SDSKNTA[B3L]WFGPRL-[NH₂]; | SEQ ID 70 |
| Hy-SDSKNTALW[B3F]GPRL-[NH₂]; | SEQ ID 71 |
| Hy-SDSKNT[Aib]LWFGPRL-[NH₂]; | SEQ ID 72 |
| Hy-SDSKNTALWFG[Hyp]RL-[NH₂]; | SEQ ID 73 |
| Hy-SDSKNT[Aib]LWFG[Hyp]RL-[NH₂]. | SEQ ID 74 |

or a salt or solvate thereof.

18. A composition, e.g. an insect control composition or plant protection composition, comprising a compound according to any one of paragraphs 1 to 17 in admixture with one or more solvents, carriers, diluents, adjuvants, preservatives, dispersants, emulsifying agents, or synergists.
19. A composition according to paragraph 18 which is an aqueous composition.
20. Use, as an insect control agent, of a compound according to any one of paragraphs 1 to 17, or a composition according to paragraph 18 or 19.
21. The use according to paragraph 20 wherein said use is as an insecticide against hemipteran, dipteran and/or lepidopteran insects.
22. A method of increasing hemipteran, dipteran and/or lepidopteran insect mortality, the method comprising contacting a hemipteran insect or hemipteran insect population with a compound according to any one of paragraphs 1 to 17, or a composition according to paragraph 18 or 19.
23. Use of a compound according to any one of paragraphs 1 to 17, or a composition according to paragraph 18 or 19, as a plant protection agent, for protecting a plant against hemipteran, dipteran and/or lepidopteran insects.
24. A method of inhibiting infestation of a plant by hemipteran, dipteran and/or lepidopteran insects comprising contacting the plant with a compound as described in any one of paragraphs 1 to 17, or a composition according to paragraph 18 or 19;
   optionally wherein the compound is applied to the plant while the plant is free or substantially free of hemipteran, dipteran and/or lepidopteran insects.
25. A method of reducing hemipteran, dipteran and/or lepidopteran insect infestation of a plant, or of reducing hemipteran, dipteran and/or lepidopteran insect load on a plant, the method comprising contacting the plant with a compound according to any one of paragraphs 1 to 17, or a composition according to paragraph 18 or 19.

1. An insecticidal compound having the formula (I) below, or a salt or solvate thereof: 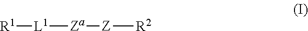
wherein:
R¹ is hydrogen (which may be designated “H—” or “Hy-”), C₁₋₄alkyl (e.g. methyl, ethyl, propyl, butyl), formyl, —N(R^{1a})—C(═N⁺(R^{1b})(R^{1c}))NR^{1d}R^{1e}, —C(═N⁺(R^{1b})(R^{1c}))NR^{1d}R^{1e}acyl, fatty acyl, benzyl, benzoyl, heteroaryl or trifluoroacetyl, —NHC₁₋₁₈alkyl, —NHC₆₋₁₆Aryl, —NH—C₁₋₆alkyl-C₆₋₁₀aryl, biotin, a sugar moiety or (poly)alkyleneglycol;
wherein each of R^{1a}, R^{1b}, R^{1c}, R^{1d} and R^{1e} is independently selected from hydrogen or C₁₋₄ alkyl (e.g. methyl, ethyl, propyl, butyl);
and any alkyl, formyl, acyl, fatty acyl, benzyl, benzoyl, heteroaryl or trifluoroacetyl, —NHC₁₋₁₈alkyl, —NHC₆₋₁₆Aryl, —NH—C₁₋₆alkyl-C₆₋₁₀aryl or (poly)alkyleneglycol may optionally be substituted with one or more groups selected from halogen, C₁₋₆alkyl, C₁₋₆haloalkyl or a sugar moeity;
L¹ is absent or is:
*—(C=O)C₁₋₁₆-alkylene-NH— where * denotes the point of attachment to Z;
(C₁₋₂₀)alkylene,
(C₂₋₂₀)alkenylene,
(poly)alkyleneglycol;
wherein L¹ is optionally substituted with one or more groups selected from oxo (═O), halogen, ═N and ═S;
Z^{a} is peptide comprising from 1 to 12 amino acids; wherein Z^{a} optionally comprises a (poly)alkyleneglycol linker in the peptide sequence;
Z is a peptide of the formula: 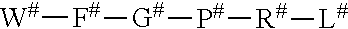
wherein “^{#}” indicates that the residues “W^{#}”, “F^{#}”, “G^{#}”, “P^{#}”, “R^{#}” and “L^{#}” are independently an unmodified amino acid, a modified amino acid or non-natural amino acid analogue; and
at least one of the residues in peptides Z^{a} and Z is a modified amino acid or non-natural amino acid analogue; and
R² is NH₂, NR^{2a}H, NR^{2a}R^{2b}, or OR^{2a}; wherein each of R^{2a} and R^{b} if present are independently C₁₋₆-alkyl (e.g. methyl, ethyl, propyl, butyl, pentyl or hexyl), C₃₋₆-alkenyl, C₆₋₁₆-aryl, C₆₋₁₆-aryl-C₁₋₆-alkyl, C₁₋₆-alkylene-C₆₋₁₆-aryl, or C₁₋₆-haloalkyl, each of which may optionally be substituted with one or more groups selected from halogen, C₁₋₆-alkyl, or C₁₋₆haloalkyl.

2. The insecticidal compound according to claim 1, wherein if R¹-L¹- is: 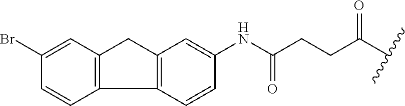 [SMILES: *C(=O)CCC(=O)Nc1ccc2c(c1)Cc1cc(Br)ccc1-2]
then at least one modified amino acid residue or non-natural amino acid residue in peptide Z^{a} or Z is not:
a beta amino acid; or
a D-configuration amino acid.

3. The insecticidal compound according to claim 1, wherein at least one modified amino acid residue or non-natural amino acid residue in peptide Z^{a} or Z is not:
a beta amino acid,
a D-configuration amino acid,
Hyp, Oic, Sar, Nle, or Aib.

4. The insecticidal compound according to any of claims 1 to 3, wherein at least one of the residues in peptides Z^{a} and Z is:
i. an N-methylated amino acid;
ii. a D-configuration amino acid;
iii. a beta-configuration amino acid
iv. a peptoid amino acid analogue;
v. a sugar modified amino acid;
vi. a biotin modified amino acid;
vii. a non-proteinogenic amino acid, such as hydroxyproline (Hyp: L-hydroxyproline or (2S,4R)-4-Hydroxyproline), Octahydroindole-2-carboxylic acid (Oic), sarcosine (Sar), norleucine (Nle), α-aminoisobutyric acid (Aib), thiazolidine-4-carboxylic acid (Thz) or phenylglycine (Phg);
optionally wherein at least one of the residues in peptides Z^{a} and Z is:
i. an N-methylated amino acid;
ii. a peptoid amino acid analogue;
iii. a sugar modified amino acid; or
iv. a biotin modified amino acid.

5. The insecticidal compound according to any one of the preceding claims, wherein at least one of the residues in peptides Z^{a} and Z is:
i. an N-methylated amino acid;
ii. a peptoid amino acid analogue;
optionally wherein at least one of the residues in peptides Z^{a} and Z is an N-methylated amino acid.

6. The insecticidal compound according to any one of the preceding claims, wherein the peptide Z comprises no more than 3 modified amino acid residues or non-natural amino acid residues; optionally wherein the peptide Z comprises no more than 2 modified amino acid residues or non-natural amino acid residues; further optionally wherein the peptide Z comprises only a single modified amino acid residue or non-natural amino acid residue.

7. The insecticidal compound according to any one of the preceding claims, wherein R¹ is selected from:
hydrogen;
acyl optionally substituted with a sugar moiety;
fatty acyl;
heteroaryl;
—NHC₆₋₁₆Aryl;
a sugar moiety;
biotin; or
(poly)ethyleneglycol of the formula *—(OCH₂CH₂)ₙ—Rₚ, where * denotes the point of attachment to L¹ or Z, n is an integer from 1 to 16, and Rₚ is selected from —NH₂, —OH or —OMe.

8. The insecticidal compound according to any one of the preceding claims, wherein R¹ is selected from:
hydrogen;
acyl optionally substituted with a sugar moiety, wherein the acyl group is selected from formyl, acetyl (Ac), propanoyl, butanoyl;
fatty acyl selected from palmitoyl, butyryl, cerotoyl, decanoyl, docosenoyl, dodecanoyl, eleostearoyl, heptanoyl, hexanoyl, icosanoyl, icosenoyl, lignoceroyl, linoleoyl, lipoyl, myristoleoyl, nonanoyl, octadecanoyl, ocatanoyl, palmitoleoyl, stearoyl, undecanoyl, and valeryl;
indolyl;
—NHC₆₋₁₆Aryl;
a monosaccharide or disaccharide moiety;
biotin;
(poly)ethyleneglycol of the formula *—(OCH₂CH₂)ₙ—Rₚ, where * denotes the point of attachment to L¹ or Z, n is an integer from 4 to 12, and Rₚ is selected from —NH₂, —OH or —OMe.

9. The insecticidal compound according to any one of the preceding claims, wherein R¹ is selected from:
hydrogen;
acetyl substituted with a monosaccharide or disaccharide moiety;
palmitoyl;
indolyl, e.g 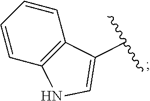 [SMILES: *c1c[nH]c2ccccc12]
—N(H)-fluorenyl substituted with one or more bromine atoms, e.g. 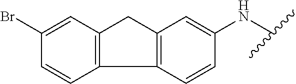 [SMILES: *Nc1ccc2c(c1)Cc1cc(Br)ccc1-2]
(poly)ethyleneglycol of the formula *—(OCH₂CH₂)ₙ—OH, where * denotes the point of attachment to Z, and n is an integer from 6 to 10.

10. The insecticidal compound according to any one of the preceding claims, wherein L¹ is absent or is:
—(C═O)C₄₋₁₂-alkylene-NH—;
—(C═O)—C₁₋₄alkylene-(C═O)—;
*—(C═O)—C₁₋₄alkylene;
where * denotes the point of attachment to Z.

11. The insecticidal compound according to any one of the preceding claims, wherein L¹ is absent or is: 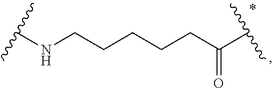 [SMILES: *NCCCCCC(*)=O]
where * denotes the point of attachment to Z; or

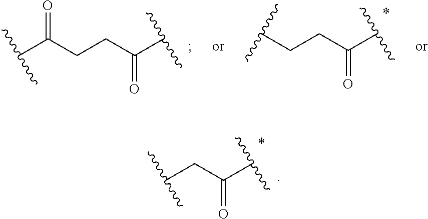 [SMILES: *C(=O)CCC(*)=O ; *CCC(*)=O ; *CC(*)=O]

12. The insecticidal compound according to any one of the preceding claims, wherein Z^{a} is a peptide of the formula: 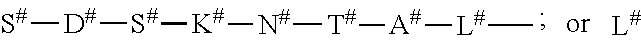
wherein “^{#}” indicates that the residue is either a naturally occurring amino acid, a modified amino acid or a non-natural amino acid analogue.

13. The insecticidal compound according to any one of the preceding claims, wherein Z^{a}—Z is a peptide of the formula: 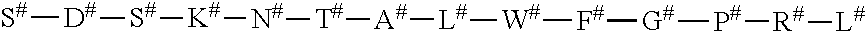
wherein one or more of the residues in Z^{a} or in Z is N-methylated;
optionally wherein the peptide Z^{a}—Z comprises 1, 2, 3 or 4 modified amino acids or non-natural amino acid analogues.

14. The insecticidal compound according to any one of the preceding claims, wherein the peptide moiety Z^{a}—Z comprises only 1, 2, 3 or 4 modified amino acids or non-natural amino acid analogues.

15. The insecticidal compound according to any one of the preceding claims, wherein the Z^{a} moiety has the formula:

| | |
|---|---|
| SDSKNTAL | SEQ ID 1 |
| SDSKNT[n-me-A]L | SEQ ID 2 |
| SDSKNT[Peptoid Phe]L | SEQ ID 3 |
| SDSKNT[Peptoid Ala]L | SEQ ID 4 |
| SDSKNTA[Peptoid Leu] | SEQ ID 5 |
| SDSKNTA[n-me-L] | SEQ ID 6 |
| [glucosamine-S]DSKNTAL | SEQ ID 7 |
| SD[glucosamine-S]KNTAL | SEQ ID 8 |
| SDSK[glucosamine-N]TAL | SEQ ID 9 |
| [glucosamine-Suc]-SDSKNTAL | SEQ ID 10 |
| [glucosamine-Suc]-SDSKNT[n-me-A]L | SEQ ID 11 |
| [Biotin]-SDSKNTAL | SEQ ID 12 |
| SDS[Biotin-K]NTAL | SEQ ID 13 |
| S[Biotin-PEG-D]SKNTAL | SEQ ID 14 |
| [Biotin]-SDSKNT[n-me-A]L | SEQ ID 15 |
| SDS[Biotin-K]NT[n-me-A]L | SEQ ID 16 |
| S[Biotin-PEG-D]SKNT[n-me-A]L | SEQ ID 17 |
| SDSKNT[Aib]L | SEQ ID 18 |
| SDSKNT[a]L; | SEQ ID 19 |
| SDSKNTA[l]; | SEQ ID 20 |
| SDSKNT[a][l] | SEQ ID 21 |
| SDSKNT[a]L | SEQ ID 22 |
| SDSKNTA[l] | SEQ ID 23 |
| SDSKNT[a][l]; | SEQ ID 24 |
| SDSKNT[B3A]L; | SEQ ID 25 |
| SDSKNTA[B3L]; | SEQ ID 26 |
| SDSKNT[Aib]L; or | SEQ ID 27 |
| L. | SEQ ID 28 |

16. The insecticidal compound according to any one of the preceding claims, wherein the Z moiety has the formula:

| | |
|---|---|
| WFGPRL; | SEQ ID 29 |
| W[Peptoid Phe]GPRL; | SEQ ID 30 |
| W[n-me-F]GPRL; | SEQ ID 31 |
| WFGPR[n-me-L]; | SEQ ID 32 |
| W[f]GPRL; | SEQ ID 33 |
| [w]FGPRL; | SEQ ID 34 |
| [w][f]GPRL; | SEQ ID 35 |
| W[B3F]GPRL; | SEQ ID 36 |
| WFG[Hyp]RL; or | SEQ ID 37 |
| W-[Phg]-[Sar]-PR-[n-me-L]. | SEQ ID 38 |

17. The insecticidal compound according to any one of the preceding claims, wherein R² is NH₂.

18. A compound according to claim 1, having the formula:

| | |
|---|---|
| Hy-Ahx-SDSKNT[n-me-A]LWFGPRL-[NH₂] | SEQ ID 39 |
| Hy-SDSKNT[n-me-A]LWFGPRL-[NH₂] | SEQ ID 40 |
| Hy-SDSKNT[Peptoid-Phe]LWFGPRL-[NH₂] | SEQ ID 152 |
| Hy-SDSKNT[Peptoid-Ala]LWFGPRL-[NH₂] | SEQ ID 41 |
| Hy-SDSKNTA[Peptoid Leu]WFGPRL-[NH₂] | SEQ ID 42 |
| Hy-SDSKNTALW[Peptoid-Phe]GPRL-[NH₂] | SEQ ID 43 |
| Hy-SDSKNTA[n-me-L]WFGPRL-[NH₂] | SEQ ID 44 |
| Hy-SDSKNTALW[n-me-F]GPRL-[NH₂] | SEQ ID 45 |
| Hy-SDSKNTALWFGPR[n-me-L]-[NH₂] | SEQ ID 46 |
| Hy-SDSKNT[n-me-A]LWFGPR[n-me-L]- | SEQ ID 47 |
| [NH₂] | |
| Hy-[Peg8]-SDSKNT[n-me-A]LWFGPRL- | SEQ ID 48 |
| [NH₂]; | |
| Hy-[Palm]-SDSKNT[n-me-A]LWFGPRL- | SEQ ID 49 |
| [NH₂]; | |
| Hy-[ado]-SDSKNT[n-me-A]LWFGPRL-[NH₂]; | SEQ ID 50 |
| Hy-[2Abf-Suc]-SDSKNT[n-me-A]LWFGPRL- | SEQ ID 51 |
| [NH₂]; | |
| [Ind]-SDSKNT[n-me-A]LWFGPRL-[NH₂]; | SEQ ID 52 |
| Hy-[glucosamine-Suc]-SDSKNT[n-me- | SEQ ID 53 |
| A]LWFGPRL-[NH₂] | |
| Hy-[biotin]-SDSKNT[n-me-A]LWFGPRL- | SEQ ID 54 |
| [NH₂] | |
| Hy-[Biotin]-SDSKNTALWFGPRL-[NH₂] | SEQ ID 55 |
| Hy-SDS[Biotin-K]NTALWFGPRL-[NH₂] | SEQ ID 56 |
| Hy-S[Biotin-PEG-D]SKNTALWFGPRL-[NH₂] | SEQ ID 57 |
| Hy-SDS[Biotin-K]NT[n-me-A]LWFGPRL- | SEQ ID 58 |
| [NH₂] | |
| Hy-S[Biotin-PEG-D]SKNT[n-me- | SEQ ID 59 |
| A]LWFGPRL-[NH₂] | |
| Hy-SDSKNT[a]LWFGPRL-[NH₂]; | SEQ ID 60 |
| Hy-SDSKNTA[l]WFGPRL-[NH₂]; | SEQ ID 61 |
| Hy-SDSKNTALW[f]GPRL-[NH₂]; | SEQ ID 62 |
| Hy-SDSKNTAL[w]FGPRL-[NH₂]; | SEQ ID 63 |
| Hy-SDSKNT[a][l]WFGPRL-[NH₂]; | SEQ ID 64 |
| Hy-SDSKNT[a]LW[f]GPRL-[NH₂]; | SEQ ID 65 |
| Hy-SDSKNTA[l]W[f]GPRL-[NH₂]; | SEQ ID 66 |
| Hy-SDSKNT[a][l]W[f]GPRL-[NH₂]; | SEQ ID 67 |
| Hy-SDSKNT[a][l][w][f]GPRL-[NH₂]; | SEQ ID 68 |
| Hy-SDSKNT[B3A]LWFGPRL-[NH₂]; | SEQ ID 69 |
| Hy-SDSKNTA[B3L]WFGPRL-[NH₂]; | SEQ ID 70 |
| Hy-SDSKNTALW[B3F]GPRL-[NH₂]; | SEQ ID 71 |
| Hy-SDSKNT[Aib]LWFGPRL-[NH₂]; | SEQ ID 72 |
| Hy-SDSKNTALWFG[Hyp]RL-[NH₂]; | SEQ ID 73 |
| Hy-SDSKNT[Aib]LWFG[Hyp]RL-[NH₂]. | SEQ ID 74 |
| Ac-LW-[Phg]-[Sar]-PR-[n-me-L]-[NH₂]; or | SEQ ID 75 |
| Hy-SDSK[glucosamine-N]TALWFGPRL- | SEQ ID 151 |
| [NH₂]; | |

or a salt or solvate thereof.

19. A composition, e.g. an insect control composition or plant protection composition, comprising a compound according to any one of claims 1 to 18 in admixture with one or more solvents, carriers, diluents, adjuvants, preservatives, dispersants, emulsifying agents, or synergists.

20. A composition according to claim 19 which is an aqueous composition.

21. Use, as an insect control agent, of a compound according to any one of claims 1 to 18, or a composition according to claim 19 or 20.

22. The use according to claim 21 wherein said use is as an insecticide against hemipteran, dipteran and/or lepidopteran insects.

23. A method of increasing hemipteran, dipteran and/or lepidopteran insect mortality, the method comprising contacting a hemipteran insect or hemipteran insect population with a compound according to any one of claims 1 to 18, or a composition according to claim 19 or 20.

24. Use of a compound according to any one of claims 1 to 18, or a composition according to claim 19 or 20, as a plant protection agent, for protecting a plant against hemipteran, dipteran and/or lepidopteran insects.

25. A method of inhibiting infestation of a plant by hemipteran, dipteran and/or lepidopteran insects comprising contacting the plant with a compound as described in any one of claims 1 to 18, or a composition according to claim 19 or 20;
optionally wherein the compound is applied to the plant while the plant is free or substantially free of hemipteran, dipteran and/or lepidopteran insects.

26. A method of reducing hemipteran, dipteran and/or lepidopteran insect infestation of a plant, or of reducing hemipteran, dipteran and/or lepidopteran insect load on a plant, the method comprising contacting the plant with a compound according to any one of claims 1 to 18, or a composition according to claim 19 or 20.
